(19) United States
(12) Patent Application Publication
(10) Pub. No.: US 2011/0047634 A1
(43) Pub. Date: Feb. 24, 2011

(54) METHODS AND COMPOSITIONS FOR THE DIAGNOSIS AND TREATMENT OF ACUTE LYMPHOBLASTIC LEUKEMIA

(75) Inventors: James Downing, Memphis, TN (US); Charles Mullighan, Memphis, TN (US)

Correspondence Address: ALSTON AND BIRD LLP;ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(73) Assignee: St. Jude Children's Research Hospital

(21) Appl. No.: 12/866,511

(22) PCT Filed: Feb. 4, 2009
(86) PCT No.: PCT/US09/33035

§ 371 Date: Nov. 12, 2010

Related U.S. Application Data
(60) Provisional application No. 61/030,679, filed on Feb. 22, 2008.

(51) Int. Cl. A01K 67/00; C07H 21/04; C07K 14/47; C12Q 1/68; C07H 21/00; C40B 30/04; G01N 33/68; G01N 33/566

(52) U.S. Cl. 800/13; 536/23.5; 530/350; 435/6; 536/24.31; 506/9; 436/86; 435/7.1; 435/7.21

(57) ABSTRACT

Compositions and methods for the identification, prognosis, classification, diagnosis, and treatment of leukemia or a genetic predisposition to leukemia are provided. The present invention is based on the discovery of a novel intragenic deletion in the v-ets erythroblastosis virus E26 oncogene homolog (ERG) allele which is shown herein to be associated with a novel subtype of B-progenitor acute lymphoblastic leukemia (ALL). In one embodiment, the intragenic deletion in ERG results in the expression of C-terminal domain deletion forms of the ERG polypeptide which lacks the DNA-binding PNT domain and CAE domain of the ERG polypeptide and have dominant negative ERG activity. In other embodiments, the intragenic deletions results in a loss of expression of the native ERG polypeptide. Such nucleotide sequences and amino acid sequences of ERG find use in methods and compositions useful in the identification and/or the prognosis and/or predisposition and/or treatment of ALL, more particularly, the novel subtype of B-progenitor AL.

### FIELD OF THE INVENTION

The present invention relates generally to the detection and treatment of a sub-type of acute lymphoblastic leukemia.

### BACKGROUND OF THE INVENTION

The leukemias comprise multiple different groups or types including, but not limited the following: acute myeloid (AML), acute lymphatic (ALL), chronic myeloid (CML) and chronic lymphatic leukemia (CLL). Within these groups, several subcategories can be identified further using a panel of standard techniques. These different subcategories of leukemia are associated with varying clinical outcome and therefore are the basis for different treatment strategies.

The development of new, specific drugs and treatment approaches requires the identification of specific subtypes that may benefit from a distinct therapeutic protocol and, thus, can improve outcome of distinct subsets of leukemia. As it is mandatory for these patients suffering from these specific leukemia subtypes to be identified as fast as possible so that the best therapy can be applied, diagnostics today must accomplish sub-classification with maximal precision. Thus, methods and compositions are needed in the art to provide means for additional leukemia diagnostics and treatment.

### SUMMARY OF THE INVENTION

Compositions and methods for the identification, prognosis, classification, diagnosis, and treatment of leukemia or a genetic predisposition to leukemia are provided. The present invention is based on the discovery of novel focal intragenic deletions of the v-ets erythroblastosis virus E26 oncogene homolog (ERG) allele which is shown herein to be associated with a novel subtype of B-progenitor acute lymphoblastic leukemia (ALL). Non-limiting examples of these intragenic deletions of ERG result in either the loss or a decrease in ERG expression or the expression of C-terminal domain deleted forms of the ERG polypeptide. Non-limiting examples of C-terminal domain deleted forms of ERG can lack the DNA-binding PNT domain and the CAE domain of the ERG polypeptide but continue to retain the ETS domain and the transactivation (TA) domain of ERG. In specific embodiments, such C-terminal domain deleted ERG polypeptides act as dominant negative forms of the normal [or wild-type] ERG polypeptide. Such nucleotide sequences and amino acid sequences of ERG find use, for example, as biomarkers for use in methods for detecting the intragenic deletions of the ERG allele which is associated with ALL, more specifically, a novel subtype of B-progenitor ALL, and thereby identifying the novel subtype of B-progenitor ALL. Further provided are methods for identifying agents that bind to and/or inhibit or decrease the activity of the C-terminal domain deleted ERG polypeptides. In addition, the C-terminal domain deleted ERG and polypeptides and polynucleotides encoding the same can serve as molecular targets for drugs useful in treating ALL, more specifically, treating a novel subtype of B-progenitor ALL. Accordingly, the present invention encompasses methods and compositions useful in the identification and/or the prognosis and/or predisposition and/or treatment of ALL, more specifically, a novel subtype of B-progenitor ALL.

### DESCRIPTION OF THE FIGURES

FIG. 1 provides a schematic illustration of the structure of the normal or wildtype ERG gene, along with the alternatively spliced ERG1 and ERG2 transcript isoforms transcribed from the wild type ERG gene. The various protein domains of the ERG polypeptide are also illustrated, as non-limiting examples of ERG intragenic deletions disclosed herein. The designation “E3” and “I#” represents the exon and intron numbers based on the genomic nomenclature. A blank box indicates that the sequence is absent.

FIG. 2 provides the amino acid alignment of the ERG polypeptide encoded by the ERG1 transcript (SEQ ID NO: 15); the C-terminal domain deleted ERG polypeptides (ERG_I1_D6-10_distal_ORF, SEQ ID NO: 16 and ERG_I1_D6-12_distal_ORF, SEQ ID NO:17); and the fusion protein predicted to be expressed from a chromosomal translocation of TMPRSS2:ERG (denoted as TMP_ERGa_ERG1_CDS, SEQ ID NO: 18).

FIG. 3 annotates the PNT domain, the CAE1 domain, the CD domain, ETS domain, and the TA domain of TMPRSS2:ERG (SEQ ID NO:18).

FIG. 4 annotates the ETS domain and the TA domain of ERG_I1_D6-10_distal_ORF_predicted protein (SEQ ID NO:16).

FIG. 5 annotates the ETS domain and the TA domain of ERG_I2_D6-12_distal_ORF_predicted protein (SEQ ID NO:20).

FIG. 6 annotates the PNT domain, the CAE1 domain, the CAE2 domain, CD domain, ETS domain, and the TA domain of ERG_isoform1_protein (SEQ ID NO:15).

FIGS. 7A and B provides the mRNA sequence of ERG_I1_D6-12 (SEQ ID NO:5). Exon boundaries are denoted.

FIG. 8 provides the mRNA sequence of ERG_I1_D6-13 (SEQ ID NO: 6). Exon boundaries are denoted.

FIG. 9 provides the mRNA sequence of ERG_I1_D6-10 (SEQ ID NO:4). Exon boundaries are denoted.

FIG. 10 provides a schematic illustrating non-limiting 5′ and 3′ genomic break points occurring in the ERG gene. SEQ ID NOS; 23-32 are shown therein.

FIG. 11 provides the annotated sequence of the native ERG isoform 1 mRNA.

FIG. 12 provides the annotated sequence of the native ERG isoform 2 mRNA.

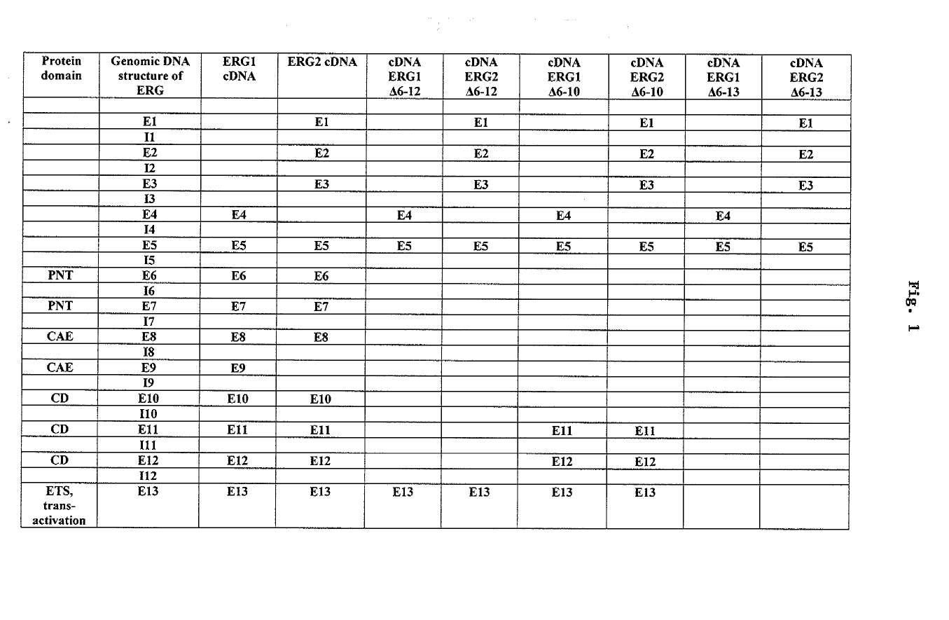

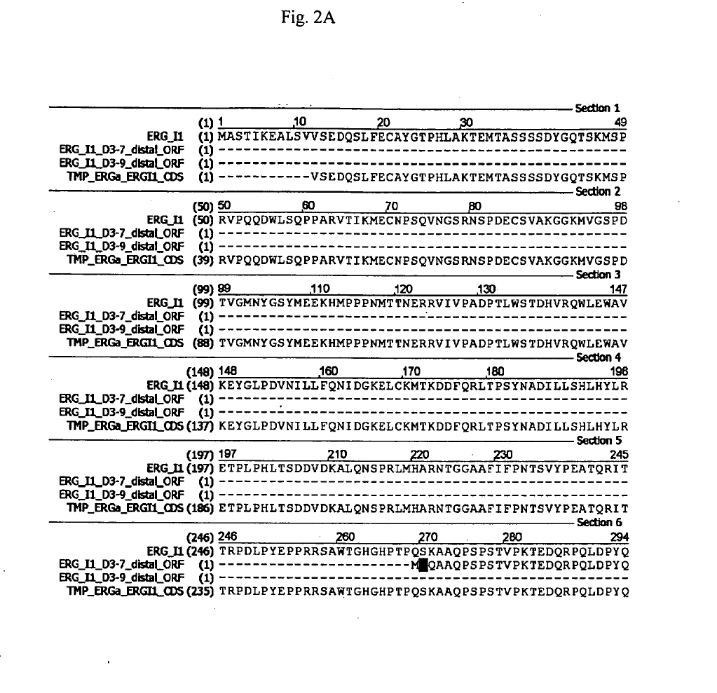

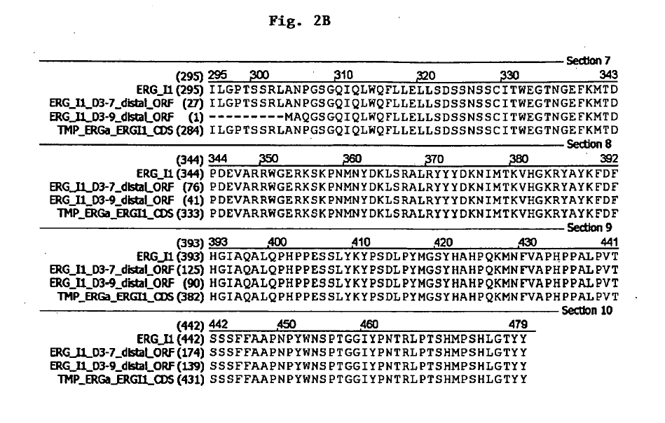

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Polynucleotide and Polypeptides

Compositions of the invention include ERG polynucleotides and polypeptides and variants and fragments thereof that are associated with acute lymphoblastic leukemia (ALL). In specific embodiments, such polynucleotides and polypeptides are associated with a subtype of B-progenitor ALL, more specifically, a novel subtype of B-progenitor ALL. As used herein, “a novel subtype of B-progenitor ALL” refers to a subtype of B-progenitor ALL characterized by a unique gene expression profile, aberrant expression of CD2, and the absence of the recurring cytogenetic abnormalities. See, for example, Yeoh et al. (2002) Cancer Cell 1:133, herein incorporated by reference in its entirety. Various methods and compositions that allow for the detection of such abnormalities in ERG are provided. Compositions of the invention include ERG polynucleotides and variants and fragments thereof that can be used to detect intragenic deletions in the ERG gene that are associated with ALL, more particularly, with a novel subtype of B-progenitor ALL.

An “ERG” polypeptide or “v-ets erythroblastosis virus E26 oncogene homolog” polypeptide refers to a transcription factor that is a member of the ETS family of transcription factors. The ERG polypeptide comprises a series of characterized domains including an ETS domain, a transactivation domain, CAE domain(s), a CD domain, and a DNA-binding PNT domain. The ETS domain is a conserved region that mediates binding to ETS motifs containing the core recognition sequence 5′-GGA(A/T)-3′ and/or engages in protein-protein interactions. Methods are known in the art to assay for the activity of the ETS domain. See, for example, Zou et al. (2005) Mol Cell Biol 25:6235-6246, herein incorporated by reference. The transactivation domain mediates DNA binding, homodimerization, heterodimerization and/or transactivation. Methods are known for assaying for the activity of the transactivation domain. See, for example, Carrere et al. (1998) Oncogene 16:3261-3268, herein incorporated by reference. The CAE (Central Alternative Exons) domain is structurally and functionally characterized. See, for example, Carrere et al. (1998) Oncogene 16:3261-3268. Methods are known in the art to assay for the activity of the CAE domain. See, for example, Carrere et al. (1998) Oncogene 3261-3268. The CD domain has a negative influence on transactivating activity. See, for example, Carrere et al. (1998) Oncogene 16:3261-3268. Methods are known in the art to assay for the activity of the CD domain. See, for example, Carrere et al. (1998) Oncogene 16:3261-3268. The DNA-binding PNT domain has DNA-binding and homodimerization activity. Methods are known in the art to assay for the activity of the DNA-binding PNT domain. See, for example, Carrere et al. (1998) Oncogene 3261-3268, herein incorporated by reference.

The human genomic sequence of ERG is set forth in SEQ ID NO:1. The various exons/introns of the ERG genomic sequence are further illustrated in the sequence listing as shown in SEQ ID NO:1. Alternative splicing allows for the production of two ERG mRNA isoforms: ERG1 (shown in SEQ ID NO: 2) and ERG2 (shown in SEQ ID NO: 3). The polypeptide encoded by the ERG1 isoform is shown in SEQ ID NO:15. It will be appreciated by those skilled in the art that DNA sequence polymorphisms may exist within a population (e.g., the human population). Such genetic polymorphisms in a polynucleotide comprising the ERG gene as set forth in SEQ ID NO:1 may exist among individuals within a population due to natural allelic variation. The term ERG gene encompasses such natural variations.

The term “gene” refers to a nucleic acid (e.g., DNA) sequence that comprises coding sequences necessary for the production of a polypeptide, precursor, or RNA (e.g., rRNA, tRNA). The term also encompasses the coding region of a structural gene and the sequences located adjacent to the coding region on both the 5′ and 3′ end which allow for the expression of the sequence. Sequences located 5′ of the coding region and present on the mRNA are referred to as 5′ non-translated sequences. Sequences located 3′ or downstream of the coding region and present on the mRNA are referred to as 3′ non-translated sequences. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “intragenic deletion” refers to any internal deletion in the genomic DNA of a gene. Thus, the term “intragenic deletion of ERG” refers to any internal deletion in the genomic DNA comprising the ERG gene. As used herein a focal intragenic deletion of an ERG allele is characterized phenotypically by the association of the intragenic deletion with a novel subtype of B-progenitor ALL. At the genetic level, the focal intragenic deletion is part of the genetic make-up of the cell (contained within the genomic DNA). In specific embodiments, the intragenic deletion of ERG can comprise a deletion of at least 1, 10, 20, 40, 80, 100, 200, 300, 400, 500, 600, 700, 800, 1000 or more nucleotides in the ERG gene. In specific embodiments, the intragenic deletion of ERG comprises an internal deletion of various exons including, for example, a deletion of at least one of exon 0, exon 1, exon 2, exon 3, exon 4, exon 5, exon 6, exon 7, exon 8, exon 9, exon 10, exon 11, exon 12, and/or exon 13 of the ERG gene or any combination thereof. It is recognized that, as used herein, a deletion of an exon or intron can encompass both the complete absence of the recited exon or intron sequence, or the absence of at least a fragment of the full exon or full intron. In other words, the chromosomal break can occur anywhere within the recited exon or in the flanking intron. The exons of the human ERG gene are designated in the genomic sequence of the human ERG gene in SEQ ID NO: 1.

The term “junction of an intragenic deletion” refers to the region of the polynucleotide which is joined following the deletion of the intervening sequences. In view of the characterization of multiple focal intragenic deletions of ERG, novel polynucleotides are provided that comprise the novel polynucleotide junctions of ERG that occur following the various focal intragenic deletions.

One of skill will recognize that while the intragenic deletion of ERG occurs in the genomic DNA, the deletions will further impact the RNA transcripts transcribed from the mutant ERG gene. Thus, the present invention provides both polynucleotides comprising the sequence of the genomic DNA having the focal intragenic deletion of ERG, and further provides polynucleotide sequences from the transcripts (partially or fully processed) or cDNAs which are derived from the focal intragenic deletion of the ERG gene. Such polynucleotides of the ERG genome or ERG transcripts are provided herein as is an extensive characterization of the novel junction sequences that occur as a result of the deletion. Thus, a polynucleotide sequence that comprises a junction of an ERG intragenic deletion can be derived from the genomic DNA or mRNA or cDNA. It is therefore recognized that the “detection” of the focal intragenic deletion of ERG can be performed by directly detecting the deletion in the genomic DNA, by directly detecting the deletion in an RNA or cDNA transcribed from the genomic DNA, or by directly detecting the truncated form of the ERG polypeptide that results from the intragenic deletion of ERG. Methods and compositions that provide for these forms of detection are discussed in further detail elsewhere herein. Non-limiting novel junctions appearing in the mRNA or cDNAs comprising the intragenic deletions are set forth in SEQ ID NO: 4, 5, 7, 8, 9, 10, 11, 12, 13, 14, 21 or 22 or variants and fragments thereof.

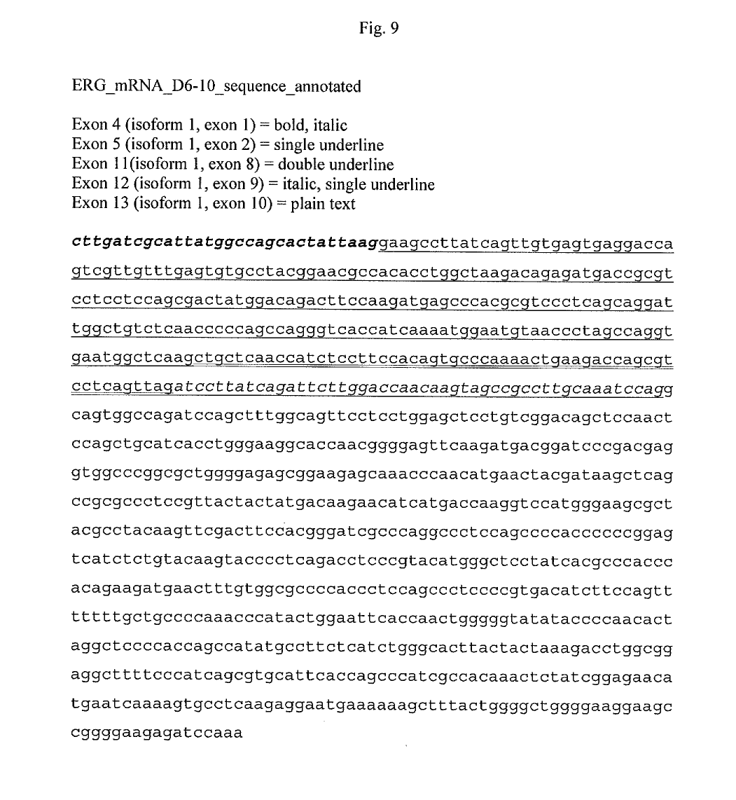

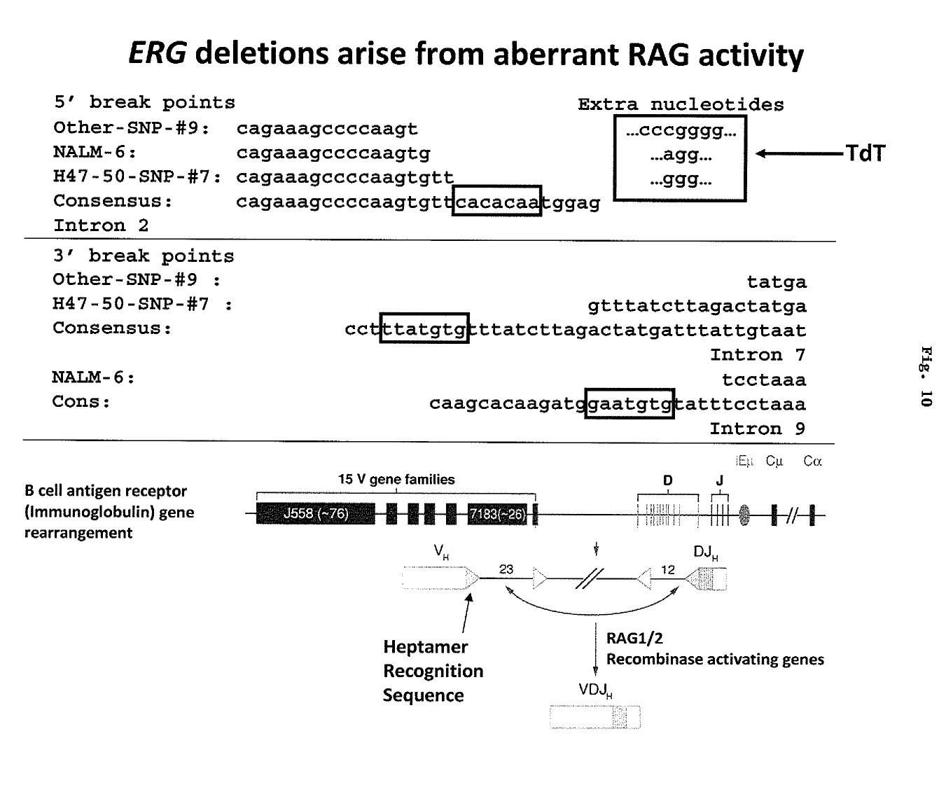

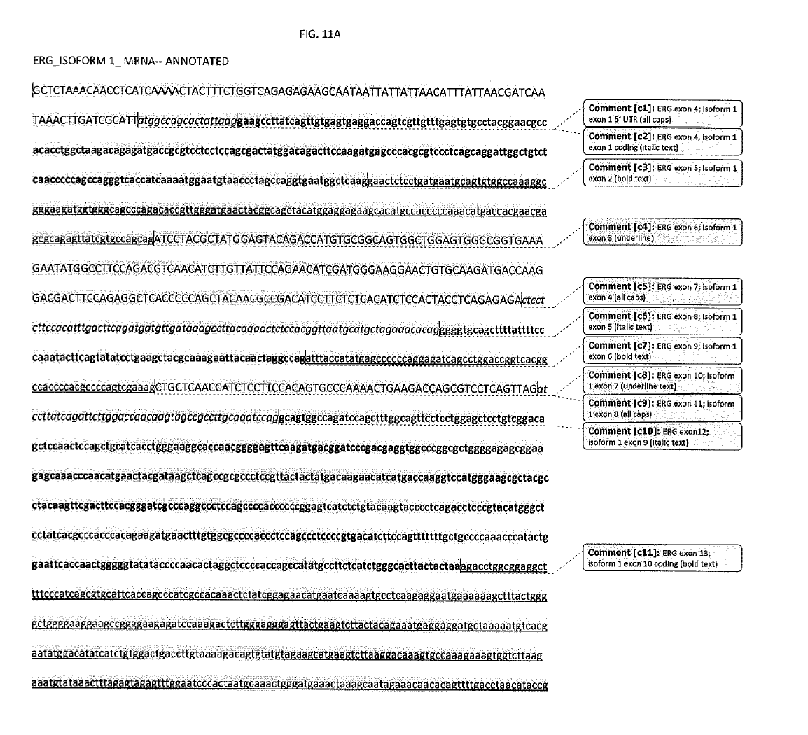

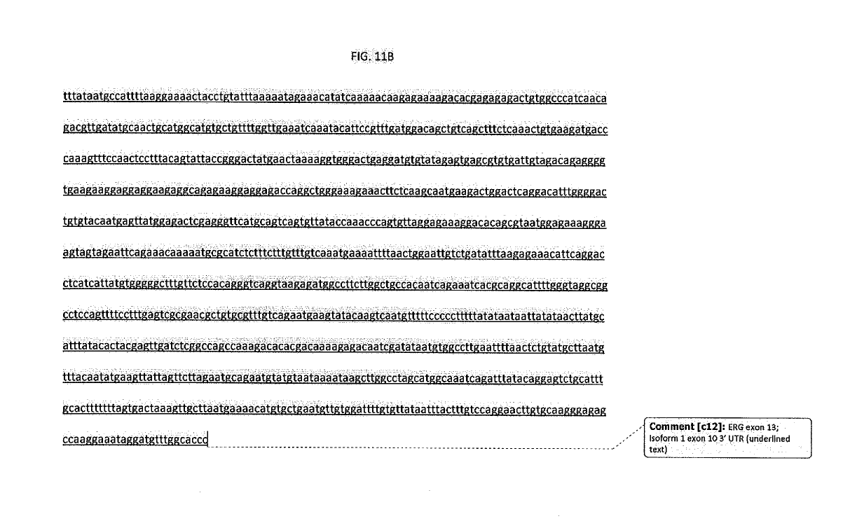

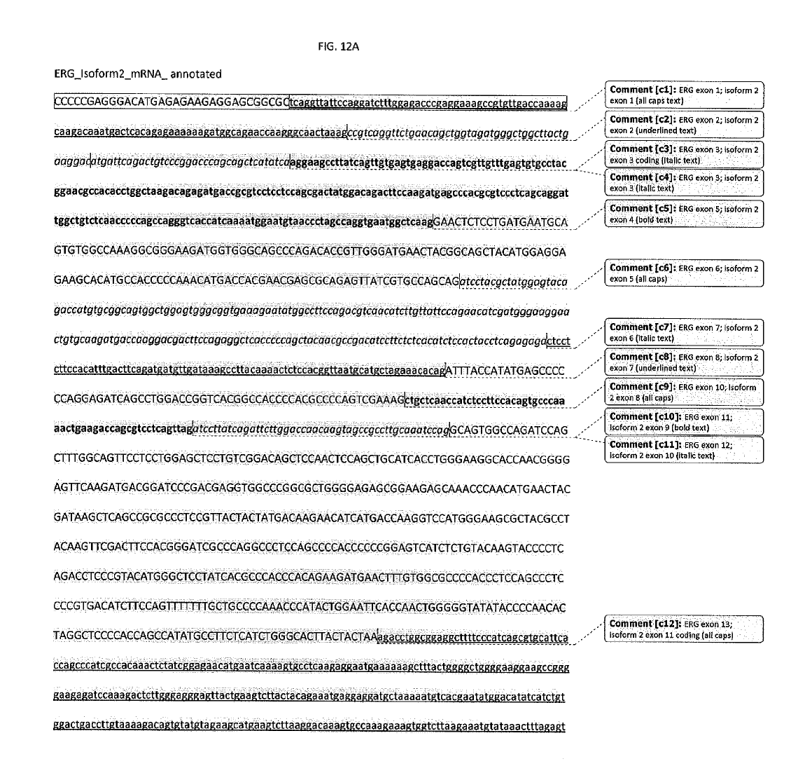

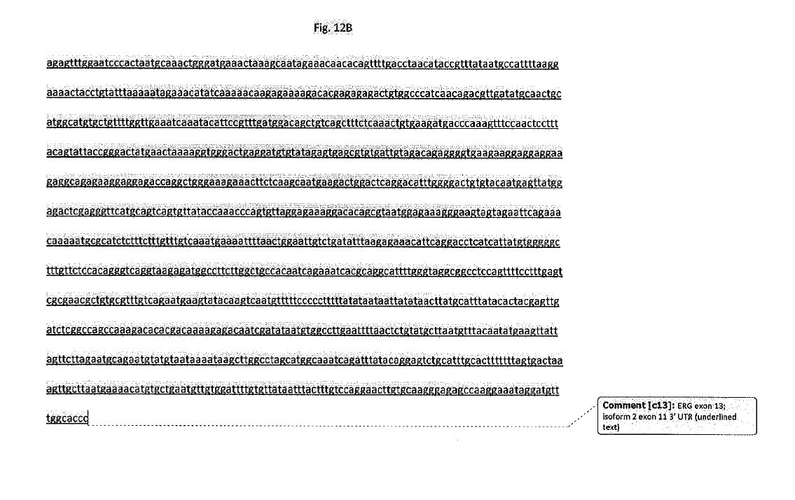

In specific embodiments, the focal intragenic deletion of ERG comprises an internal deletion of various exons including, for example, a deletion of exon 6 through exon 10. In further embodiments, the genomic abnormality resulting in the deletion of exon 6 through exon 10 results from a proximal chromosomal break point occurring within intron 5 and a distal chromosomal break point occurring within intron 10. See, for example, SEQ ID NO:1. This specific genomic abnormality is referred to herein as ERGΔexon6-10. The cDNA derived from isoform 1 of the ERGΔexon6-10 intragenic deletion is set forth in SEQ ID NO:4 and shown in FIG. 9. The genomic sequence of ERG Δexon6-10 is shown in SEQ ID NO: 19. The various intron and exon boundaries are denoted.

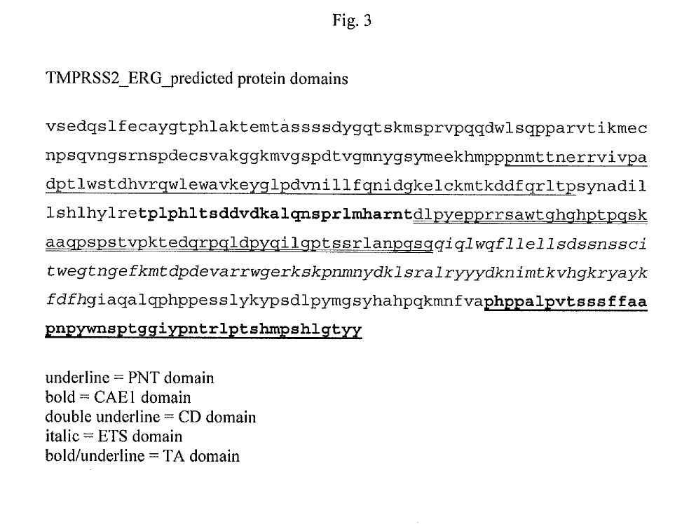

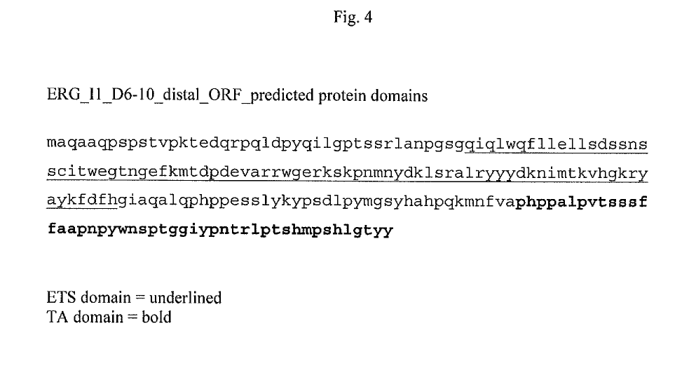

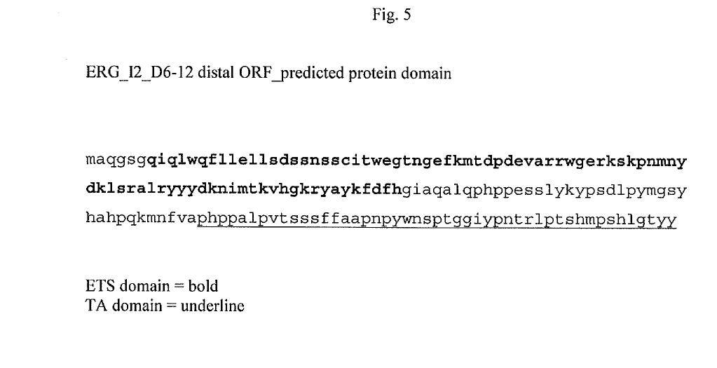

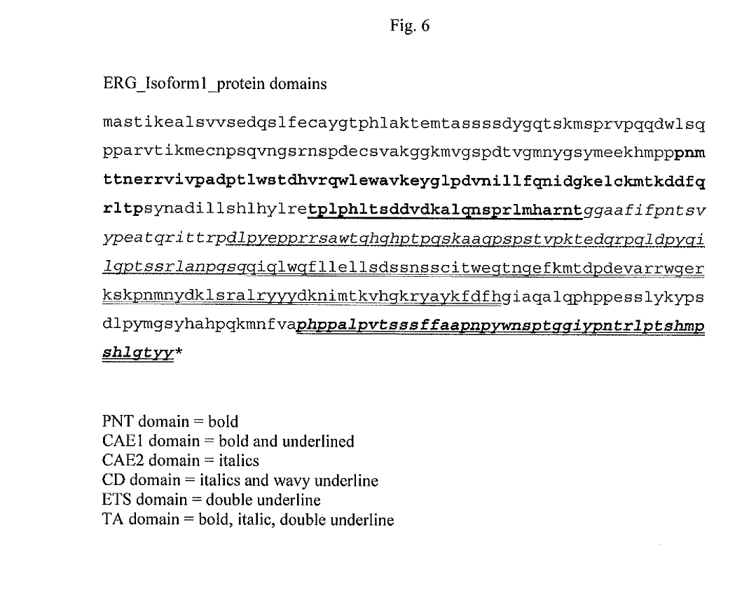

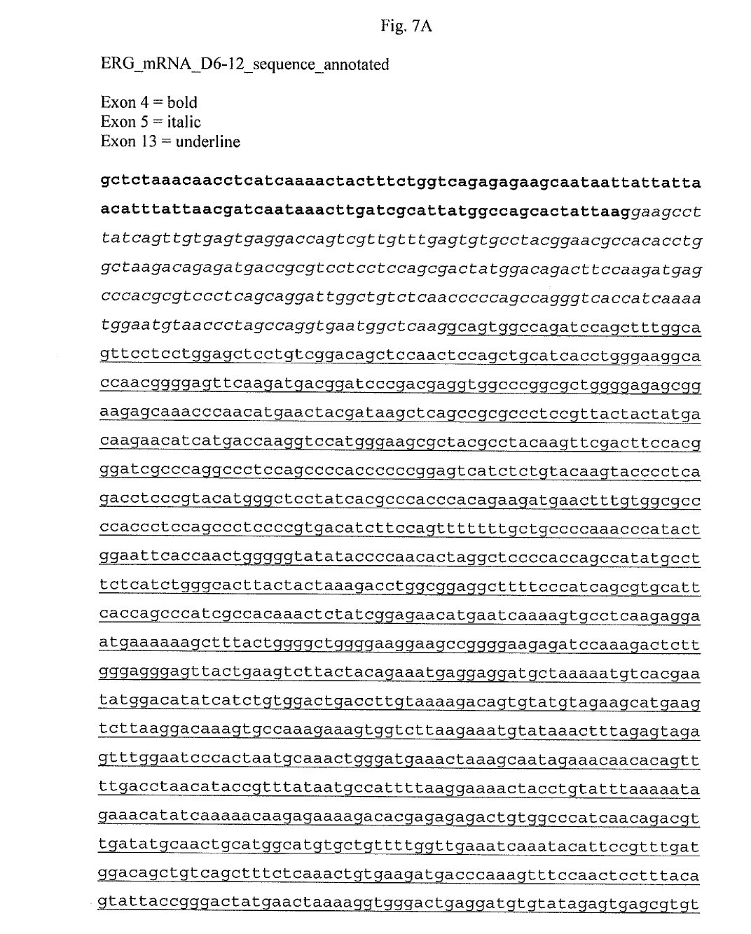

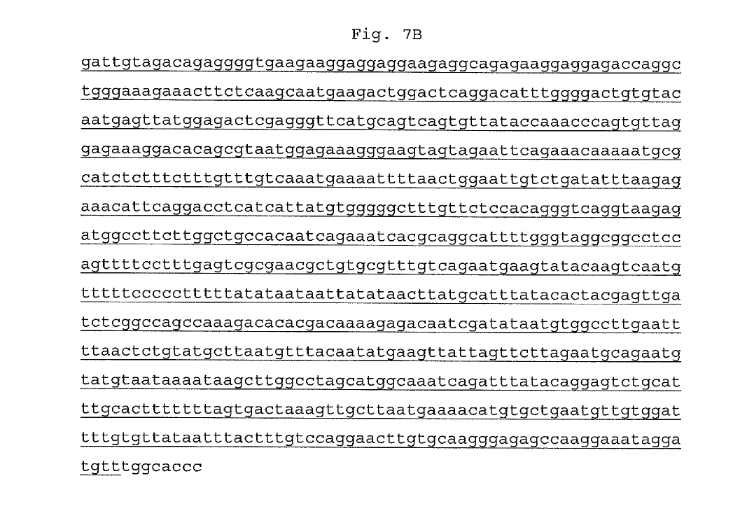

In other embodiments, the focal intragenic deletion of ERG comprises an internal deletion of various exons including, for example, a deletion of exon 6 through exon 12. In further embodiments, the genomic abnormality resulting in the deletion of exon 6 through exon 12 results from a proximal chromosomal break point occurring within intron 5 and a distal chromosomal break point occurring within intron 12. See, for example, FIG. 3. This specific genomic abnormality is referred to herein as EEGΔexon6-12. The cDNA derived from isoform 1 of the EEGΔexon6-12 intragenic deletion is set forth in SEQ ID NO:5 and shown in FIG. 7.

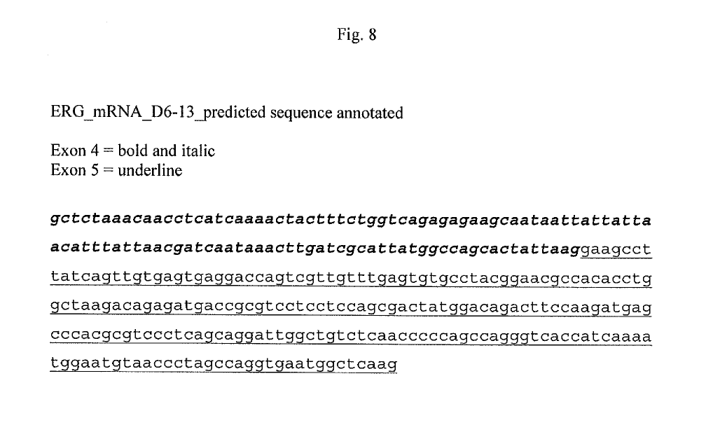

In specific embodiments, the focal intragenic deletion of ERG comprises an internal deletion of various exons including, for example, a deletion of exon 6 through exon 13. In further embodiments, the genomic abnormality resulting in the deletion of exon 6 through exon 13 results from a proximal chromosomal break point occurring within intron 5 and a distal chromosomal break point in the 3′ untranslated region of ERG. See, for example, FIG. 3. This specific genomic abnormality is referred to herein as EEGΔexon6-13. The predicted cDNA derived from isoform 1 of the EEGΔexon6-13 intragenic deletion is set forth in SEQ ID NO:6 and shown in FIG. 8. Such sequences are predicted to be hypomorphic and not act as a competitor (or dominant negative inhibitor) of normal ERG.

In specific embodiments, the polynucleotides comprising the junctions of the ERG intragenic deletion or active variants and fragments thereof, do not encode an ERG polypeptide, but rather have the ability to specifically detect the ERG intragenic deletion in the nucleic acid complement of a biological sample, and thereby allow for the identification and/or classification and/or the prognosis and/or predisposition of the biological sample to ALL, more particularly, a novel subtype of B-progenitor ALL. Various methods and compositions to carry out such methods are disclosed elsewhere herein.

Alternatively, the polynucleotides comprising the junctions of the ERG intragenic deletion or active variants and fragments thereof, encode a C-terminal domain deleted ERG polypeptide or active variant or fragment thereof. As used herein, a “C-terminal deleted domain or fragment thereof” comprises any of the following domains (or fragments of the domains) of the ERG protein: PNT domain, CAE domain, CD domain, ETS domain and TA domain. A “C-terminal domain deleted polypeptide” comprises any ERG polypeptide that lacks at least one of these domains or at least a fragment of one of these domains.

As discussed in further detail herein, in specific embodiments, the C-terminal domain deleted ERG polypeptides of the invention do not comprises a DNA binding PNT domain or a CAE domain. Thus, in specific embodiments, the C-terminal domain deleted ERG polypeptide comprises the CD domain and the ETS, transactivation domain and does not comprise the DNA binding PNT domain and CAE domain. See, for example, ERG_D6-10_distal ORF set forth in SEQ ID NO: 16. In other embodiments, the C-terminal domain deleted ERG polypeptide comprises a fragment of the CD domain and the ETS, transactivation domain and does not comprise the DNA binding PNT domain or CAE domain. See, for example, ERG_D6-12_distal ORF as set forth in SEQ ID NO:17. In specific embodiments, the C-terminal domain deleted ERG polypeptides have dominant negative ERG activity.

As used herein, “dominant negative ERG activity” of a C-terminal domain deleted ERG polypeptide comprises an activity that inhibits the activity of the wild type form of the ERG polypeptide. Such activity can be assayed for using various methods known in the art, including for example, Luciferase reporter assays of ERG transactivating activity. See, for example, Carrere et al (1998) Oncogene 16:3261 and Zou et al (2005) Mol Cell Biol 25:6235, both of which are incorporated by reference. Alternatively, the dominate negative activity can be assessed by assaying for a phenotype of leukemia, and is specific embodiments, a novel subtype of B-progenitor ALL.

In other embodiments, the intragenic deletion of ERG results in a decreased level of expression of the ERG polypeptide. A decreased level of expression of the ERG polypeptide results in at least about a 10%, about a 20%, about a 30%, about a 40%, about a 50%, about a 60%, about a 70%, about a 80%, about a 90%, about a 95% or about a 100% loss of expression of the ERG polypeptide. The decreased level of expression can occur at any stage in the processing and synthesis of protein, including, for example, during transcription or translation. In other embodiments, a decreased level of the activity of the ERG polypeptide can occur and results in at least about a 10%, about a 20%, about a 30%, about a 40%, about a 50%, about a 60%, about a 70%, about a 80%, about a 90%, about a 95% or about a 100% loss of ERG polypeptide activity.

In specific embodiments, detecting the ERG intragenic deletions find use in selecting a therapy for a subject affect by leukemia. Thus, upon the detection of the ERG intragenic deletion, and in specific embodiments, the identification of the specific ERG intragenic deletion, a therapy may be selected or customized for the subject in view of the ERG intragenic deletion.

### II. Methods of Detecting ERG Intragenic Deletions

a. Detecting Polynucleotides

Various methods and compositions for identifying a genomic abnormality in the ERG gene are provided. Such methods find use in identifying and/or detecting such rearrangements in any biological material and thus allow for the identification, prognosis, classification, treatment, and/or diagnosis of leukemia or a genetic predisposition to ALL, more particularly, to the novel subtype of B-progenitor ALL.

In one embodiment, a method is provided for assaying a biological sample for an intragenic deletion of the ERG gene. The method comprises (a) providing a biological sample from a subject, wherein the biological sample comprises the nucleic acid complement of the subject and (b) determining if the nucleic acid complement comprises an intragenic deletion in the ERG gene. In such a method, the presence of the intragenic deletion of the ERG gene is indicative of ALL, more particularly, the novel subtype of B-progenitor ALL.

Such methods can be used to identify the various ERG intragenic deletions discussed above, including for example, a deletion of at least one exon of the ERG gene or a deletion that results in the decreased expression of the ERG. In specific methods, the ERG intragenic deletion that is detected comprises a deletion of exon 6 through exon 10 of the ERG gene; a deletion of exon 6 through exon 12 of the ERG gene; or a deletion of exon 6 through exon 13 of the ERG gene.

It is further recognized that the diagnostic method used to detect the intragenic deletion may be one which allows for the detection of the deletion without discriminating between the various ERG intragenic deletions disclosed herein. Alternatively, the method employed may be such as to allow for a specific ERG intragenic deletion to be distinguished. In other methods, an initial assay may be performed to confirm the presence of an ERG intragenic deletion but not identify the specific deletion. If desired, a secondary assay can then performed to determine the identity of the particular ERG intragenic deletion. The second assay may use a different detection technology than the initial assay.

It is further recognized that the ERG intragenic deletion may be detected along with other markers in a multiplex or panel format. Markers are selected for their predictive value alone or in combination with the ERG intragenic deletion. Markers for other leukemias, diseases, infections, and metabolic conditions are also contemplated for inclusion in a multiplex of panel format. Ultimately, the information provided by the methods of the present invention will assist a physician in choosing the best course of treatment for a particular patient.

As used herein, the use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

As used herein, the “nucleic acid complement” of a sample comprises any polynucleotide contained in the sample. The nucleic acid complement that is employed in the methods and compositions of the invention can include all of the polynucleotides contained in the sample or any fraction thereof. For example, the nucleic acid complement could comprise the genomic DNA and/or the mRNA and/or cDNAs of the given biological sample. Thus, the intragenic deletion of ERG can be detected in the genomic DNA or through the transcribed products thereof.

As used herein, a “biological sample” can comprise any sample in which one desires to determine if the nucleic acid complement of the sample contains an ERG intragenic deletion. For example, a biological sample can comprise a sample from any organism, including a mammal, such as a human, a primate, a rodent, a domestic animal (such as a feline or canine) or an agricultural animal (such as a ruminant, horse, swine or sheep). The biological sample can be derived from any cell, tissue or biological fluid from the organism of interest. The sample may comprises any clinically relevant tissue, such as, but not limited to, bone marrow samples, tumor biopsy, fine needle aspirate, or a sample of bodily fluid, such as, blood, plasma, serum, lymph, ascitic fluid, cystic fluid or urine. In other embodiments, the biological sample comprises peripheral blood, bone marrow, apheresis samples, cerebrospinal fluid, saliva, urine, gonadal tissue, tissue (e.g. chloroma) biopsies, or any other human tissue sample potentially involved by leukemic infiltration. The sample used in the methods of the invention will vary based on the assay format, nature of the detection method, and the tissues, cells or extracts which are used as the sample. It is recognized that the sample typically requires preliminary processing designed to isolate or enrich the sample for the genomic DNA. A variety of techniques known to those of ordinary skill in the art may be used for this purpose.

As used herein, a “probe” is an isolated polynucleotide to which is attached a conventional detectable label or reporter molecule, e.g., a radioactive isotope, ligand, chemiluminescent agent, enzyme, etc. Such a probe is complementary to a strand of a target polynucleotide, which in specific embodiments of the invention comprise a polynucleotide comprising a junction of the ERG intragenic deletion. If the novel junction is in the transcribed mRNA, such probes can comprise the polynucleotide set forth in any one of SEQ ID NOS: 4, 5, 7, 8, 9, 10, 11, 12, 13, 14, 19, 21, 22 or a variant or fragment thereof. Deoxyribonucleic acid probes may include those generated by PCR using ERG specific primers, oligonucleotide probes synthesized in vitro, or DNA obtained from bacterial artificial chromosome, fosmid or cosmid libraries. Probes include not only deoxyribonucleic or ribonucleic acids but also polyamides and other probe materials that can specifically detect the presence of the target DNA sequence. For nucleic acid probes, examples of detection reagents include, but are not limited to radiolabeled probes, enzymatic labeled probes (horse radish peroxidase, alkaline phosphatase), affinity labeled probes (biotin, avidin, or steptavidin), and fluorescent labeled probes (6-FAM, VIC, TAMRA, MGB, fluorescein, rhodamine, texas red [for BAC/fosmids]). One skilled in the art will readily recognize that the nucleic acid probes described in the present invention can readily be incorporated into one of the established kit formats which are well known in the art.

As used herein, “primers” are isolated polynucleotides that are annealed to a complementary target DNA strand by nucleic acid hybridization to form a hybrid between the primer and the target DNA strand., then extended along the target DNA strand by a polymerase, e.g., a DNA polymerase. Primer pairs of the invention refer to their use for amplification of a target polynucleotide, e.g., by the polymerase chain reaction (PCR) or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

Probes and primers are of sufficient nucleotide length to bind to the target DNA sequence and specifically detect and/or identify a polynucleotide comprising an ERG intragenic deletion or a junction of an ERG intragenic deletion. It is recognized that the hybridization conditions or reaction conditions can be determined by the operator to achieve this result. This length may be of any length that is of sufficient length to be useful in a detection method of choice. Generally, 8, 11, 14, 16, 18, 20, 22, 24, 26, 28, 30, 40, 50, 75, 100, 200, 300, 400, 500, 600, 700 nucleotides or more, or between about 11-20, 20-30, 30-40, 40-50, 50-100, 100-200, 200-300, 300-400, 400-500, 500-600, 600-700, 700-800, or more nucleotides in length are used. Such probes and primers can hybridize specifically to a target sequence under high stringency hybridization conditions. Probes and primers according to embodiments of the present invention may have complete DNA sequence identity of contiguous nucleotides with the target sequence, although probes differing from the target DNA sequence and that retain the ability to specifically detect and/or identify a target DNA sequence may be designed by conventional methods. Accordingly, probes and primers can share about 80%, 85%, 90%, 91%, 92%, 93%, 94%, 95%, 96%, 97%, 98%, 99% or greater sequence identity or complementarity to the target polynucleotide (i.e., SEQ ID NO: 1, 2, 3, 4, 5, 6, 7, 8, 9, 10, 11, 12, 13, 14, 19, 21, 22, or to a fragment thereof). Probes can be used as primers, but are generally designed to bind to the target DNA or RNA and are not used in an amplification process.

Specific primers can be used to amplify the junction of an ERG intragenic deletion to produce an amplicon that can be used as a “specific probe” or can itself be detected for identifying an ERG intragenic deletion in a biological sample. When the probe is hybridized with the polynucleotides of a biological sample under conditions which allow for the binding of the probe to the sample, this binding can be detected and thus allow for an indication of the presence of the ERG intragenic deletion in the biological sample. Such identification of a bound probe has been described in the art. The specific probe may comprise a sequence of at least 80%, between 80 and 85%, between 85 and 90%, between 90 and 95%, and between 95 and 100% identical (or complementary) to a specific region of the ERG gene or cDNA.

As used herein, “amplified DNA” or “amplicon” refers to the product of polynucleotide amplification of a target polynucleotide that is part of a nucleic acid template. For example, to determine whether the nucleic acid complement of a biological sample comprises an ERG intragenic deletion, the nucleic acid complement of the biological sample may be subjected to a polynucleotide amplification method using a primer pair that includes a first primer derived from the 5′ flanking sequence adjacent to a junction of an ERG intragenic deletion, and a second primer derived from the 3′ flanking sequence adjacent to the junction of the ERG intragenic deletion to produce an amplicon that is diagnostic for the presence of the ERG intragenic deletion. By “diagnostic” for an ERG intragenic deletion is intended the use of any method or assay which discriminates between the presence or absence of an ERG intragenic deletion in a biological sample. The amplicon is of a length and has a sequence that is also diagnostic for the ERG intragenic deletion (i.e., has a junction sequence of the ERG intragenic deletion). The amplicon may range in length from the combined length of the primer pairs plus one nucleotide base pair to any length of amplicon producible by a DNA amplification protocol. A member of a primer pair derived from the flanking sequence may be located a distance from the junction or breakpoint. This distance can range from one nucleotide base pair up to the limits of the amplification reaction, or about twenty thousand nucleotide base pairs. The use of the term “amplicon” specifically excludes primer dimers that may be formed in the DNA thermal amplification reaction.

Methods for preparing and using probes and primers are described, for example, in Molecular Cloning: A Laboratory Manual, 2.sup.nd ed, vol. 1-3, ed. Sambrook et al., Cold Spring Harbor Laboratory Press, Cold Spring Harbor, N.Y. 1989 (hereinafter, “Sambrook et al., 1989”); Current Protocols in Molecular Biology, ed. Ausubel et al., Greene Publishing and Wiley-Interscience, New York, 1992 (with periodic updates) (hereinafter, “Ausubel et al., 1992”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using guidelines known to one of skill in the art.

As outline in further detail below, any conventional nucleic acid hybridization or amplification or sequencing method can be used to specifically detect the presence of a polynucleotide arising due to an ERG intragenic deletion. By “specifically detect” is intended that the polynucleotide can be used either as a primer to amplify the junction of an ERG intragenic deletion or the polynucleotide can be used as a probe that hybridizes under stringent conditions to a polynucleotide having an ERG intragenic deletion. The level or degree of hybridization which allows for the specific detection of the ERG intragenic deletion is sufficient to distinguish the polynucleotide with the ERG intragenic deletion from a polynucleotide that does not contain the deletion and thereby allow for discriminately identifying an ERG intragenic deletion. By “shares sufficient sequence identity or complementarity to allow for the amplification of an ERG intragenic deletion” is intended the sequence shares at least 80%, 85%, 90%, 91%, 92%, 93%, 94%, 95%, 96%, 97%, 98%, 99% or 100% identity or complementarity to a fragment or across the full length of the ERG polynucleotide.

The ERG intragenic deletion may be detected using a variety of nucleic acid techniques known to those of ordinary skill in the art, including but not limited to: nucleic acid sequencing; nucleic acid hybridization; and, nucleic acid amplification. Nucleic acid hybridization includes methods using labeled probes directed against purified DNA, amplified DNA, and fixed leukemia cell preparations (fluorescence in situ hybridization).

Illustrative non-limiting examples of nucleic acid sequencing techniques include, but are not limited to, chain terminator (Sanger) sequencing and dye terminator sequencing. Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom. Dye terminator sequencing alternatively labels the terminators. Complete sequencing can be performed in a single reaction by labeling each of the di-deoxynucleotide chain-terminators with a separate fluorescent dye, which fluoresces at a different wavelength.

The present invention further provides methods for identifying nucleic acids containing an ERG intragenic deletion which do not necessarily require sequence amplification and are based on, for example, the known methods of Southern (DNA:DNA) blot hybridizations, in situ hybridization and FISH of chromosomal material, using appropriate probes. Such nucleic acid probes can be used that comprise nucleotide sequences in proximity to the ERG intragenic deletion junction, or breakpoint. By “in proximity to” is intended within about 100 kilobases (kb) of the ERG intragenic deletion junction.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts. In some embodiments, the ERG intragenic deletion is detected using fluorescence in situ hybridization (FISH).

In specific embodiments, probes for detecting an ERG intragenic deletion are labeled with appropriate fluorescent or other markers and then used in hybridizations. The Examples section provided herein sets forth various protocol that are effective for detecting the genomic abnormalities, but one of skill in the art will recognize that many variations of these assay can be used equally well. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: hi Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo et al. (1991) Am. J. Hum. Genet. 42:112-119; Klinger et al. (1992) Am. J. Hum. Genet. 51:55-65; and Ward et al. (1993) Am. J. Hum. Genet. 52:854-865). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

Southern blotting can be used to detect specific DNA sequences. In such methods, DNA that is extracted from a sample is fragmented, electrophoretically separated on a matrix gel, and transferred to a membrane filter. The filter bound DNA is subject to hybridization with a labeled probe complementary to the sequence of interest. Hybridized probe bound to the filter is detected.

In hybridization techniques, all or part of a polynucleotide that selectively hybridizes to a target polynucleotide having an ERG intragenic deletion is employed. By “stringent conditions” or “stringent hybridization conditions” when referring to a polynucleotide probe is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing). Generally, a probe is less than about 1000 nucleotides in length or less than 500 nucleotides in length.

As used herein, a substantially identical or complementary sequence is a polynucleotide that will specifically hybridize to the complement of the nucleic acid molecule to which it is being compared under high stringency conditions. Appropriate stringency conditions which promote DNA hybridization, for example, 6× sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaC1/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In hybridization reactions, specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.) and Haymes et al. (1985) In: Nucleic Acid Hybridization, a Practical Approach, IRL Press, Washington, D.C.

A polynucleotide is said to be the “complement” of another polynucleotide if they exhibit complementarity. As used herein, molecules are said to exhibit “complete complementarity” when every nucleotide of one of the polynucleotide molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions.

Regarding the amplification of a target polynucleotide (e.g., by PCR) using a particular amplification primer pair, “stringent conditions” are conditions that permit the primer pair to hybridize to the target polynucleotide to which a primer having the corresponding sequence (or its complement) would bind and preferably to produce an identifiable amplification product (the amplicon) having a junction of an ERG intragenic deletion in a DNA thermal amplification reaction. In a PCR approach, oligonucleotide primers can be designed for use in PCR reactions to amplify a junction of an ERG intragenic deletion. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It is understood that a number of parameters in a specific PCR protocol may need to be adjusted to specific laboratory conditions and may be slightly modified and yet allow for the collection of similar results. These adjustments will be apparent to a person skilled in the art.

The amplified polynucleotide (amplicon) can be of any length that allows for the detection of the ERG intragenic deletion. For example, the amplicon can be about 10, 50, 100, 200, 300, 500, 700, 100, 2000, 3000, 4000, 5000 nucleotides in length or longer.

Any primer can be employed in the methods of the invention that allows a junction of the ERG intragenic deletion to be amplified and/or detected. Methods for designing PCR primers are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Other known methods of PCR that can be used in the methods of the invention include, but are not limited to, methods using paired primers, nested primers, single specific primers, degenerate primers, gene-specific primers, mixed DNA/RNA primers, vector-specific primers, partially mismatched primers, and the like.

Thus, in specific embodiments, a method of detecting the presence of an ERG intragenic deletion in a biological sample is provided. The method comprises (a) providing a sample comprising the nucleic acid complement of a subject; (b) providing a pair of DNA primer molecules that can amplify an amplicon having a junction of an ERG intragenic deletion (c) providing DNA amplification reaction conditions; (d) performing the DNA amplification reaction, thereby producing a DNA amplicon molecule; and (e) detecting the DNA amplicon molecule. In order for a nucleic acid molecule to serve as a primer or probe it need only be sufficiently complementary in sequence to be able to form a stable double-stranded structure under the particular solvent and salt concentrations employed.

In still other embodiments, the intragenic deletion of ERG may be amplified prior to or simultaneous with detection. Illustrative non-limiting examples of nucleic acid amplification techniques include, but are not limited to, polymerase chain reaction (PCR), ligase chain reaction (LCR), strand displacement amplification (SDA), and nucleic acid sequence based amplification (NASBA). The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. For other various permutations of PCR see, e.g., U.S. Pat. Nos. 4,683,195, 4,683,202 and 4,800,159; Mullis et al, (1987) Meth. Enzymol. 155: 335; and, Murakawa et al., (1988) DNA 7: 287, each of which is herein incorporated by reference in its entirety.

The ligase chain reaction (Weiss (1991) Science 254: 1292, herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker et al. (1992) Proc. Natl. Acad. Sci. USA 89: 392-396; U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTP[alpha]S to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Non-amplified or amplified ERG intragenic deletions can be detected by any conventional means. For example, the intragenic deletions can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Nelson et al. (1995) Nonisotopic Probing, Blotting, and Sequencing, ch. 17 (Larry J. Kricka ed., 2d ed., each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

Various methods can be used to detect the ERG intragenic deletion or amplicon having a junction of an ERG intragenic deletion, including, but not limited to, Genetic Bit Analysis (Nikiforov et al. (1994) Nucleic Acid Res. 22: 4167-4175) where a DNA oligonucleotide is designed which overlaps both the adjacent flanking DNA sequence and the inserted DNA sequence. The oligonucleotide is immobilized in wells of a microwell plate. Following PCR of the region of interest (using one primer in the inserted sequence and one in the adjacent flanking sequence) a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another detection method is the Pyrosequencing technique as described by Winge ((2000) Innov. Pharma. Tech. 00: 18-24). In this method, an oligonucleotide is designed that overlaps the junction. The oligonucleotide is hybridized to a single-stranded PCR product from the region of interest (one primer in the inserted sequence and one in the flanking sequence) and incubated in the presence of a DNA polymerase, ATP, sulfurylase, luciferase, apyrase, adenosine 5′ phosphosulfate and luciferin. dNTPs are added individually and the incorporation results in a light signal which is measured. A light signal indicates the presence of the transgene insert/flanking sequence due to successful amplification, hybridization, and single or multi-base extension.

Fluorescence Polarization as described by Chen et al. ((1999) Genome Res. 9: 492-498, 1999) is also a method that can be used to detect an amplicon of the invention. Using this method, an oligonucleotide is designed which overlaps the inserted DNA junction. The oligonucleotide is hybridized to a single-stranded PCR product from the region of interest (one primer in the inserted DNA and one in the flanking DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the genomic abnormality sequence due to successful amplification, hybridization, and single base extension.

Taqman® (PE Applied Biosystems, Foster City, Calif.) is described as a method of detecting and quantifying the presence of a DNA sequence and is fully understood in the instructions provided by the manufacturer. Briefly, a FRET oligonucleotide probe is designed which overlaps the junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

In one embodiment, the method of detecting an intragenic deletion of ERG comprises (a) contacting the biological sample with a polynucleotide probe that hybridizes under stringent hybridization conditions with a polynucleotide having an ERG intragenic deletion and specifically detects the ERG intragenic deletion; (b) subjecting the sample and probe to stringent hybridization conditions; and (c) detecting hybridization of the probe to the polynucleotide, wherein detection of hybridization indicates the presence of the ERG intragenic deletion.

B. Detecting Polypeptides

ERG polypeptides expressed from the ERG gene having the intragenic deletions, including for example, the C-terminal domain deleted ERG polypeptides, may be detected using a variety of protein techniques known to those of ordinary skill in the art, including but not limited to protein sequencing and immunoassays.

Illustrative non-limiting examples of protein sequencing techniques include, but are not limited to, mass spectrometry and Edman degradation. Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the C-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., calorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

### III. Kits

The materials used in the above assay methods are ideally suited for the preparation of a kit. Various detection reagents can be developed and used to assay the presence of the ERG intragenic deletion. The terms “kits” and “systems,” as used herein in the context of the ERG intragenic deletion detection reagents, are intended to refer to such things as combinations of multiple ERG intragenic deletion detection reagents, or one or more ERG intragenic deletion detection reagents in combination with one or more other types of elements or components (e.g., other types of biochemical reagents, containers, packages, such as packaging intended for commercial sale, substrates to which SNP detection reagents are attached, electronic hardware components, and the like). Accordingly, the present invention further provides ERG intragenic deletion detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more ERG intragenic deletion. The kits/systems can optionally include various electronic hardware components. For example, arrays (e.g., DNA chips) and microfluidic systems (e.g., lab-on-a-chip systems) provided by various manufacturers typically include hardware components. Other kits/systems (e.g., probe/primer sets) may not include electronic hardware components, but can include, for example, one or more ERG intragenic deletion detection reagents along with other biochemical reagents packaged in one or more containers.

In some embodiments, an ERG intragenic deletion kit typically contains one or more detection reagents and other components (e.g., a buffer, enzymes, such as DNA polymerases or ligases, chain extension nucleotides, such as deoxynucleotide triphosphates, positive control sequences, negative control sequences, and the like) necessary to carry out an assay or reaction, such as amplification and/or detection of a polynucleotide comprising a junction of one of the ERG intragenic deletion. A kit can further contain means for determining the amount of the target polynucleotide and means for comparing with an appropriate standard, and can include instructions for using the kit to detect the ERG intragenic deletion. In one embodiment, kits are provided which contain the necessary reagents to carry out one or more assays to detect one or more of the ERG intragenic deletion as disclosed herein. The ERG intragenic deletion detection kits/systems may contain, for example, one or more probes, or pairs of probes, that hybridize to a nucleic acid molecule at or near the junction region.

In specific embodiments, a kit for identifying an ERG intragenic deletion in a biological sample is provided. The kit comprises a first and a second primer, wherein the first and second primer amplify a polynucleotide comprising an ERG intragenic deletion junction and thereby detect an ERG intragenic deletion.

Further provided are polynucleotide detection kits comprising at least one polynucleotide that can specifically detect an ERG intragenic deletion. In specific embodiments, the polynucleotide comprises at least one polynucleotide molecule of a sufficient length of contiguous nucleotides homologous or complementary to SEQ ID NO: 1, 2, 3, 4, 5, 6, 7, 8, 9, 10, 11, 12, 13, 14, 19, 21, 22 or a variant thereof to allow for the detection of an ERG intragenic deletion.

Kits can also be used to detect the polypeptide expressed from the ERG gene having the intragenic deletion. In specific embodiments, a C-terminal domain deleted ERG polypeptide is detected. C-terminal domain deleted ERG detection reagents, are intended to refer to such things as at least one C-terminal domain deleted ERG polypeptide detection reagent, or one or more C-terminal domain deleted ERG polypeptide detection reagents in combination with one or more other types of elements or components (e.g., other types of biochemical reagents, containers, packages, such as packaging intended for commercial sale, electronic hardware components, and the like).

For antibody based detection systems, the present invention provides a kit which comprises an antibody capable of specifically binding to a C-terminal domain deleted ERG polypeptide and one or more of the following: wash reagents and reagents capable of detecting the presence of bound antibodies of the kit.

In specific embodiments, the kit comprises a compartmentalized kit and includes any kit in which reagents are contained in separate containers. Such containers include small glass containers, plastic containers or strips of plastic or paper. Such containers allow one to efficiently transfer reagents from one compartment to another compartment such that the samples and reagents are not cross-contaminated, and the agents or solutions of each container can be added in a quantitative fashion from one compartment to another. Such containers may include a container which will accept the test sample, a container which contains the antibodies or probes used in the assay, containers which contain wash reagents (such as phosphate buffered saline, Tris-buffers, etc.), and containers which contain the reagents used to detect the bound antibody or the hybridized probe. Any detection reagents known in the art can be used including, but not limited to those described supra.

### IV. Compounds Useful in Modulating the Activity of a C-Terminal Domain Deleted ERG Polypeptide

Further provided are methods for identifying agents that target a C-terminal domain deleted ERG polypeptide. Thus, methods to screen for compounds that can serve as molecular targets for drugs useful in modulating the activity or the level of expression of a C-terminal domain deleted ERG polypeptide are provided. Such compounds can find use in treating, preventing and/or delaying progression of ALL, more specifically, a novel subtype of B-progenitor ALL. The invention provides a method (also referred to herein as a “screening assay”) for identifying modulators, i.e., candidate or test compounds or agents (e.g., peptides, peptidomimetics, small molecules, nucleic acids or other drugs) that modulate (e g inhibits) the activity of a C-terminal domain deleted ERG polypeptide or decrease the level of a nucleic acid molecule encoding the C-terminal domain deleted ERG polypeptide.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

The compounds screened in the above assay can be, but are not limited to, small molecules, peptides, carbohydrates, nucleic acids, or vitamin derivatives. The agents can be selected and screened at random or rationally selected or designed using protein modeling techniques. For random screening, agents such as peptides or carbohydrates are selected at random and are assayed for their ability to bind to the C-terminal domain deleted ERG polypeptide or that target the nucleic acid encoding the C-terminal domain deleted ERG polypeptide. Alternatively, agents may be rationally selected or designed. As used herein, an agent is said to be “rationally selected or designed” when the agent is chosen based on the configuration of the C-terminal domain deleted ERG polypeptide. For example, one skilled in the art can readily adapt currently available procedures to generate peptides capable of binding to a specific peptide sequence in order to generate rationally designed antipeptide peptides, see, for example, Hurby et al., “Application of Synthetic Peptides: Antisense Peptides,” in Synthetic Peptides: A User's Guide, W. H. Freeman, New York (1992), pp. 289-307; and Kaspczak et al., Biochemistry 28:9230-2938 (1989).

Determining the ability of the test compound to specifically bind to a C-terminal domain deleted ERG polypeptide can be accomplished, for example, by coupling the test compound with a radioisotope or enzymatic label such that binding of the test compound to the C-terminal domain deleted ERG polypeptide can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In another embodiment, an assay of the present invention is a cell-free assay comprising contacting an C-terminal domain deleted ERG polypeptide with a test compound and determining the ability of the test compound to specifically bind to the C-terminal domain deleted ERG polypeptide. Binding of the test compound to the C-terminal domain deleted ERG polypeptide can be determined either directly or indirectly as described above.

In another embodiment, an assay is a cell-free assay comprising contacting the C-terminal domain deleted ERG polypeptide with a test compound and determining the ability of the test compound to specifically inhibit the activity of the C-terminal domain deleted ERG polypeptide. Determining the ability of the test compound to inhibit the activity of the C-terminal domain deleted ERG polypeptide using any method that can assay for the dominant negative ERG activity. Such assays are discussed elsewhere herein. In addition, one could assay for the treatment, prevention and delayed progression of ALL, more specifically, the novel subtype of B-progenitor ALL.

Such desired compounds may be further screened for selectivity by determining whether they suppress or eliminate phenotypic changes or activities associated with expression of the C-terminal domain deleted ERG polypeptide in the cells. The agents are screened by administering the agent to the cell or alternatively, the activity of the selective agent can be monitored in an in vitro assay. It is recognized that it is preferable that a range of dosages of a particular agent be administered to the cells to determine if the agent is useful for treating, preventing or delaying the onset or progression of ALL, more particularly, the novel subtype of B-progenitor ALL.

There are numerous variations of the above assays which can be used by a skilled artisan in order to isolate agonists. See, for example, Burch, R. M., in Medications Development. Drug Discovery, Databases, and Computer-Aided Drug Design, NIDA Research Monograph 134, NIH Publication No. 93-3638, Rapaka, R. S., and Hawks, R. L., eds., U.S. Dept. of Health and Human Services, Rockville, Md. (1993), pages 37-45.

Using the above procedures, the present invention provides a compound capable of binding or modulating the activity of a C-terminal domain deleted ERG polypeptide, produced by a method comprising the steps of (a) contacting the compound with the C-terminal domain deleted ERG polypeptide, and (b) determining whether the agent binds or modulates the activity of the C-terminal domain deleted ERG polypeptide. Additional step(s) to determine whether such binding is selective (i.e., does not interfere or minimally interferes with the activity of the wild-type ERG polypeptide) for the C-terminal domain deleted ERG polypeptide may also be employed. Thus, the methods can be used to detect agents that bind and/or modulate the activity of the native/wild type ERG and the C-terminal domain deleted ERG polypeptides or the methods can be used to detect agents that specifically bind and/or modulate the activity of the C-terminal domain deleted ERG polypeptides.

As used herein, an “ERG target sequence” comprises any ERG sequence that one desires to decrease the level of expression. In specific embodiments, the ERG target sequence encodes a C-terminal ERG truncate polypeptide. By “reduces” or “reducing” the expression level of a polynucleotide or a polypeptide encoded thereby is intended to mean, the polynucleotide or polypeptide level of the ERG target sequence is statistically lower than the polynucleotide level or polypeptide level of the same target sequence in an appropriate control which is not exposed to the silencing element. In particular embodiments, reducing the polynucleotide level and/or the polypeptide level of the target sequence according to the presently disclosed subject matter results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control. Methods to assay for the level of the RNA transcript, the level of the encoded polypeptide, or the activity of the polynucleotide or polypeptide are discussed elsewhere herein. Thus, the present invention provides methods and compositions to reduce the level of expression of an ERG intragenic deletion by introducing into a cell expressing the ERG intragenic deletion a silencing element which reduces or eliminates the level of expression of an ERG target polynucleotide or the polypeptide encoded thereby.

As used herein, the term “silencing element” refers to a polynucleotide comprising or encoding an interfering RNA that is capable of reducing or eliminating the level of expression of an ERG target polynucleotide or the polypeptide encoded thereby. The term “interfering RNA” or “RNAi” refers to any RNA molecule which can enter an RNAi pathway and thereby reduce the level of a target polynucleotide of interest or reduce the level of expression of a polynucleotide of interest. RNAi is distinct from so-called “antisense” mechanisms that typically involve inhibition of a target transcript by a single-stranded oligonucleotide through RNase H mediated pathway. See Crooke (ed.) (2001) “Antisense Drug Technology: Principles, Strategies, and Applications,” 1st ed., (Marcel Dekker; ISBN: 0824705661).

Thus, a silencing element can comprise the interfering RNA, a precursor to the interfering RNA, a template for the transcription of an interfering RNA or a template for the transcription of a precursor interfering RNA, wherein the precursor is processed within the cell to produce an interfering RNA. Thus, for example, a dsRNA silencing element includes a dsRNA molecule, a transcript or polyribonucleotide capable of forming a dsRNA, more than one transcript or polyribonucleotide capable of forming a dsRNA, a DNA encoding dsRNA molecule, or a DNA encoding one strand of a dsRNA molecule. When the silencing element comprises a DNA molecule encoding an interfering RNA, it is recognized that the DNA can be transiently expressed in a cell or stably incorporated into the genome of the cell. Such methods are discussed in further detail elsewhere herein.

The silencing element can reduce or eliminate the expression level of a target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, e.g., Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional interfering RNA that are capable of reducing or eliminating the level of a sequence of interest are disclosed elsewhere herein.

Any region of the target ERG polynucleotide having the intragenic deletion can be used to design a domain of the silencing element that shares sufficient sequence identity to allow for the silencing element to decrease the level of the target polynucleotide. In specific embodiments, expression of the silencing element reduces the level of the ERG sequence having the intragenic deletion (i.e., a sequence encoding the ERG C-terminal domain deleted polypeptide), while not reducing or only minimally reducing the level of the native or wild type ERG sequence. For instance, the silencing element can be designed to share sequence identity to novel junctions of the ERG intragenic deletions. Such sequences are disclosed elsewhere herein. See, for example, SEQ ID NO:4-14, 19, 21, and 22. It is recognized that a given silencing element can share about 80%, 85%, 90%, 91%, 92%, 93%, 94%, 95%, 96%, 97%, 98%, 99% or greater sequence identity or complementarity to the target polynucleotide.

The ability of a silencing element to reduce the level of the ERG target polynucleotide may be assessed directly by measuring the amount of the target transcript using, for example, Northern blots, nuclease protection assays, reverse transcription (RT)-PCR, real-time RT-PCR, microarray analysis, and the like. Alternatively, the ability of the silencing element to reduce the level of the target polynucleotide may be measured directly using a variety of affinity-based approaches including, but not limited to, Western blots, immunoassays, ELISA, flow cytometry, protein microarrays, and the like. In still other methods, the ability of the silencing element to reduce the level of the target polynucleotide can be assessed indirectly, e.g., by measuring a functional activity of the polypeptide encoded by the transcript or by measuring a signal produced by the polypeptide encoded by the transcript.

A silencing element can be prepared according to any available technique including, but not limited to, chemical synthesis, enzymatic or chemical cleavage in vivo or in vitro, template transcription in vivo or in vitro, or combinations of the foregoing.

Representative types of silencing elements are discussed in further detail below.

i. Double Stranded RNA Silencing Elements

In one embodiment, the silencing element comprises or encodes a double stranded RNA molecule. As used herein, a “double stranded RNA” or “dsRNA” refers to a polyribonucleotide structure formed either by a single self-complementary RNA molecule or a polyribonucleotide structure formed by the expression of least two distinct RNA strands. Accordingly, as used herein, the term “dsRNA” is meant to encompass other terms used to describe nucleic acid molecules that are capable of mediating RNA interference or gene silencing, including, for example, small RNA (sRNA), short-interfering RNA (siRNA), double-stranded RNA (dsRNA), micro-RNA (miRNA), hairpin RNA, short hairpin RNA (shRNA), and others. See, e.g., Meister and Tuschl (2004) Nature 431:343-349 and Bonetta et al. (2004) Nature Methods 1:79-86.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand,” and the strand homologous to the target polynucleotide is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. For example, the hairpin RNA molecule that hybridizes with itself to form a hairpin structure can comprises a single-stranded loop region and a base-paired stem. The base-paired stem region can comprise a sense sequence corresponding to all or part of the target polynucleotide and further comprises an antisense sequence that is fully or partially complementary to the sense sequence. Thus, the base-paired stem region of the silencing element can determine the specificity of the silencing. See, e.g., Chuang and Meyerowitz (2000) Proc. Natl. Acad. Sci. USA 97:4985-4990, herein incorporated by reference. A transient assay for the efficiency of hpRNA constructs to silence gene expression in vivo has been described by Panstruga et al. (2003) Mol. Biol. Rep. 30:135-140, herein incorporated by reference.

ii. siRNA Silencing Elements

A “short interfering RNA” or “siRNA” comprises an RNA duplex (double-stranded region) and can further comprises one or two single-stranded overhangs, e.g., 3′ or 5′ overhangs. The duplex can be approximately 19 base pairs (bp) long, although lengths between 17 and 29 nucleotides, including 17, 18, 19, 20, 21, 22, 23, 24, 25, 26, 27, 28, and 29 nucleotides, can be used. An siRNA can be formed from two RNA molecules that hybridize together or can alternatively be generated from a single RNA molecule that includes a self-hybridizing portion. The duplex portion of an siRNA can include one or more bulges containing one or more unpaired and/or mismatched nucleotides in one or both strands of the duplex or can contain one or more noncomplementary nucleotide pairs. One strand of an siRNA (referred to herein as the antisense strand) includes a portion that hybridizes with a target transcript. In certain embodiments, one strand of the siRNA (the antisense strand) is precisely complementary with a region of the target transcript over at least about 17 nucleotides, 18 nucleotides, 19 nucleotides, 20 nucleotides or more meaning that the siRNA antisense strand hybridizes to the target transcript without a single mismatch (i.e., without a single noncomplementary base pair) over that length. In other embodiments, one or more mismatches between the siRNA antisense strand and the targeted portion of the target transcript can exist. In embodiments in which perfect complementarity is not achieved, any mismatches between the siRNA antisense strand and the target transcript can be located at or near 3′ end of the siRNA antisense strand. For example, in certain embodiments, nucleotides 1-9, 2-9, 2-10, and/or 1-10 of the antisense strand are perfectly complementary to the target.

Considerations for design of effective siRNA molecules are discussed in McManus et al. (2002) Nature Reviews Genetics 3: 737-747 and in Dykxhoorn et al. (2003) Nature Reviews Molecular Cell Biology 4: 457-467. Such considerations include the base composition of the siRNA, the position of the portion of the target transcript that is complementary to the antisense strand of the siRNA relative to the 5′ and 3′ ends of the transcript, and the like. A variety of computer programs also are available to assist with selection of siRNA sequences, e.g., from Ambion (web site having URL www.ambion.com), at web site having URL www.sinc.sunysb.edu/Stu/shilin/rnai.html. Additional design considerations that also can be employed are described in Semizarov et al. Proc. Natl. Acad. Sci. 100: 6347-6352.

iii. Short Hairpin RNA Silencing Elements

The term “short hairpin RNA” or “shRNA” refers to an RNA molecule comprising at least two complementary portions hybridized or capable of hybridizing to form a double-stranded (duplex) structure sufficiently long to mediate RNAi (generally between approximately 17 and 29 nucleotides in length, including 17, 18, 19, 20, 21, 22, 23, 24, 25, 26, 27, 28, and 29 nucleotides in length, and in some embodiments, typically at least 19 base pairs in length), and at least one single-stranded portion, typically between approximately 1 and 20 or 1 to 10 nucleotides in length that forms a loop connecting the two nucleotides that form the base pair at one end of the duplex portion. The duplex portion can, but does not require, one or more bulges consisting of one or more unpaired nucleotides. In specific embodiments, the shRNAs comprise a 3′ overhang. Thus, shRNAs are precursors of siRNAs and are, in general, similarly capable of inhibiting expression of a target transcript.

In particular, RNA molecules having a hairpin (stem-loop) structure can be processed intracellularly by Dicer to yield an siRNA structure referred to as short hairpin RNAs (shRNAs), which contain two complementary regions that hybridize to one another (self-hybridize) to form a double-stranded (duplex) region referred to as a stem, a single-stranded loop connecting the nucleotides that form the base pair at one end of the duplex, and optionally an overhang, e.g., a 3′ overhang. The stem can comprise about 19, 20, or 21 by long, though shorter and longer stems (e.g., up to about 29 nt) also can be used. The loop can comprise about 1-20, including 1, 2, 3, 4, 5, 6, 7, 8, 9, 10, 11, 12, 13, 14, 15, 16, 17, 18, 19, 20 nt, about 4-10, or about 6-9 nt. The overhang, if present, can comprise approximately 1-20 nt or approximately 2-10 nt. The loop can be located at either the 5′ or 3′ end of the region that is complementary to the target transcript whose inhibition is desired (i.e., the antisense portion of the shRNA).

Although shRNAs contain a single RNA molecule that self-hybridizes, it will be appreciated that the resulting duplex structure can be considered to comprise sense and antisense strands or portions relative to the target mRNA and can thus be considered to be double-stranded. It will therefore be convenient herein to refer to sense and antisense strands, or sense and antisense portions, of an shRNA, where the antisense strand or portion is that segment of the molecule that forms or is capable of forming a duplex with and is complementary to the targeted portion of the target polynucleotide, and the sense strand or portion is that segment of the molecule that forms or is capable of forming a duplex with the antisense strand or portion and is substantially identical in sequence to the targeted portion of the target transcript. In general, considerations for selection of the sequence of the antisense strand of an shRNA molecule are similar to those for selection of the sequence of the antisense strand of an siRNA molecule that targets the same transcript.

iv. MicroRNA Silencing Elements

In one embodiment, the silencing element comprises an miRNA. MicroRNAs” or “miRNAs” are regulatory agents comprising about 19 ribonucleotides which are highly efficient at inhibiting the expression of target polynucleotides. See, e.g., Saetrom et al. (2006) Oligonucleotides 16:115-144, Wang et al. (2006) Mol. Cell 22:553-60, Davis et al. (2006) Nucleic Acid Research 34:2294-304, Pasquinelli (2006) Dev. Cell 10:419-24, all of which are herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

It is recognized that various forms of an miRNA can be transcribed including, for example, the primary transcript (termed the “pri-miRNA”) which is processed through various nucleolytic steps to a shorter precursor miRNA (termed the “pre-miRNA”); the pre-miRNA; or the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (McManus et al. (2002) RNA 8:842-50). In specific embodiments, 2-8 nucleotides of the miRNA are perfectly complementary to the target. A large number of endogenous human miRNAs have been identified. For structures of a number of endogenous miRNA precursors from various organisms, see Lagos-Quintana et al. (2003) RNA 9(2):175-9; see also Bartel (2004) Cell 116:281-297.

A miRNA or miRNA precursor can share at least about 80%, 85%, 90%, 91%. 92%, 93%, 94%, 95%, 96%, 97%, 98%, 99%, or 100% sequence complementarity with the target transcript for a stretch of at least about 15, 16, 17, 18, 19, 20, 21, 22, 23, 24, 25, 26, 27, 28, 29, or 30 nucleotides. In specific embodiments, the region of precise sequence complementarity is interrupted by a bulge. See Ruvkun (2001) Science 294: 797-799, Zeng et al. (2002) Molecular Cell 9:1-20, and Mourelatos et al. (2002) Genes Dev 16:720-728.

### V. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

An “isolated” or “purified” polynucleotide or polypeptide or biologically active fragment or variant thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated nucleic acid molecules can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

By “fragment” is intended a portion of the polynucleotide or a portion of the amino acid sequence and hence protein encoded thereby. Fragments of a polynucleotide may encode protein fragments that retain the biological activity of the dominant negative form of the ERG polypeptide. Alternatively, fragments of a polynucleotide that comprise junction polynucleotides of an intragenic deletion of ERG are useful as, for example, probes and primers and need not encode the ERG polypeptide. Instead, such fragments and variants are able to detect an intragenic deletion in ERG that is associated with ALL. Thus, fragments of a nucleotide sequence may range from at least about 10, about 15, 20 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length polynucleotide employed in the invention. Methods to assay for the activity of a desired polynucleotide or polypeptide are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides employed in the invention. Variant polynucleotides also include synthetically derived polynucleotide, such as those generated, for example, by using site-directed mutagenesis, but continue to retain the desired activity. Generally, variants of a particular polynucleotide of the invention having the desired activity will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides employed in the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the subject polypeptide by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of protein, as discussed elsewhere herein. Such variants may result from, for example, genetic polymorphism or from human manipulation. Biologically active variants of a native protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

In addition to the various ERG polynucleotides comprising the junctions of the intragenic deletion as shown in SEQ ID NOS:4, 5, 6, 7, 8, 9, 10, 11, 12, 13, 14, 19, 21, or 22 it will be appreciated by those skilled in the art that DNA sequence polymorphisms may exist within a population (e.g., the human population). Such genetic polymorphism in a polynucleotide comprising the junction of the intragenic deletion of ERG may exist among individuals within a population due to natural allelic variation. An allele is one of a group of genes that occur alternatively at a given genetic locus.

### VI. Expression Cassettes and Host Cells

An expression cassette comprises one or more regulatory sequences, selected on the basis of the cells to be used for expression, operably linked to the desired polynucleotide. “Operably linked” is intended to mean that the desired polynucleotide (i.e., a DNA encoding a silencing element, DNA encoding a polypeptide that increases homologous recombination activity, DNA that encodes a sequence that decreases non-homologous recombination, selectable markers, etc.) is linked to the regulatory sequence(s) in a manner that allows for expression of the nucleotide sequence (e.g., in an in vitro transcription/translation system or in a cell when the expression cassette or vector is introduced into a cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells, those that direct expression of the nucleotide sequence only in certain host cells (e.g., tissue-specific regulatory sequences), or those that direct expression of the polynucleotide in the presence of an appropriate inducer (inducible promoter). It will be appreciated by those skilled in the art that the design of the expression cassette can depend on such factors as the choice of the host cell to be transformed, the level of expression of the polynucleotide that is desired, and the like. Such expression cassettes typically include one or more appropriately positioned sites for restriction enzymes, to facilitate introduction of the nucleic acid into a vector.

It will further be appreciated that appropriate promoter and/or regulatory elements can readily be selected to allow expression of the relevant transcription units in the cell of interest. In certain embodiments, the promoter utilized to direct intracellular expression of a silencing element is a promoter for RNA polymerase III (Pol III). References discussing various Pol III promoters, include, for example, Yu et al. (2002) Proc. Natl. Acad. Sci. 99(9), 6047-6052; Sui et al. (2002) Proc. Natl. Acad. Sci. 99(8), 5515-5520 (2002); Paddison et al. (2002) Genes and Dev. 16, 948-958; Brummelkamp et al. (2002) Science 296, 550-553; Miyagashi (2002) Biotech. 20, 497-500; Paul et al. (2002) Nat. Biotech. 20, 505-508; Tuschl et al. (2002) Nat. Biotech. 20, 446-448. According to other embodiments, a promoter for RNA polymerase I, e.g., a tRNA promoter, can be used. See McCown et al. (2003) Virology 313(2):514-24; Kawasaki (2003) Nucleic Acids Res. 31 (2):700-7.

The regulatory sequences can also be provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

Various constitutive promoters are known. For example, in various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest. Promoters which may be used include, but are not limited to, the long terminal repeat as described in Squinto et al. (1991) Cell 65:1 20); the SV40 early promoter region (Bernoist and Chambon (1981) Nature 290:304 310), the CMV promoter, the M-MuLV 5′ terminal repeat the promoter contained in the 3′ long terminal repeat of Rous sarcoma virus (Yamamoto et al. (1980) Cell 22:787 797), the herpes thymidine kinase promoter (Wagner et al. (1981) Proc. Natl. Acad. Sci. U.S.A. 78:144 1445), the regulatory sequences of the metallothionine gene (Brinster et al. (1982) Nature 296:39 42); the following animal transcriptional control regions, which exhibit tissue specificity and have been utilized in transgenic animals: elastase I gene control region which is active in pancreatic acinar cells (Swift et al. (1984) Cell 38:639 646; Ornitz et al. (1986) Cold Spring Harbor Symp. Quant. Biol. 50:399 409; MacDonald, 1987, Hepatology Z:425 515); insulin gene control region which is active in pancreatic beta cells (Hanahan (1985) Nature 315:115 122), immunoglobulin gene control region which is active in lymphoid cells (Grosschedl et al. (1984) Cell 38:647 658; Adames et al (1985) Nature 318:533 538; Alexander et al. (1987) Mol. Cell. Biol. 7:1436 1444), mouse mammary tumor virus control region which is active in testicular, breast, lymphoid and mast cells (Leder et al. (1986) Cell 45:485 495).

Inducible promoters are also known. Non-limiting examples of inducible promoters and their inducer inlcude MT II/Phorbol Ester (TPA) (Palmiter et al. (1982) Nature 300:611) and heavy metals (Haslinger and Karin (1985) Proc. Nat'l Acad. Sci. USA. 82:8572; Searle et al. (1985) Mol. Cell. Biol. 5:1480; Stuart et al. (1985) Nature 317:828; Imagawa et al. (1987) Cell 51:251; Karin et al. (1987) Mol. Cell Biol. 7:606; Angel et al. (1987) Cell 49:729; McNeall et al. (1989) Gene 76:8); MMTV (mouse mammary tumor virus)/Glucocorticoids (Huang et al. (1981) Cell 27:245; Lee et al. (1981) Nature 294:228; Majors and Varmus (1983) Proc. Nat'l Acad. Sci. USA. 80:5866; Chandler et al. (1983) Cell 33:489; Ponta et al. (1985) Proc. Nat'l Acad. Sci. USA. 82:1020; Sakai et al. (1988) Genes and Dev. 2:1144); β-Interferon/poly(rI)X and poly(rc) (Tavernier et al. (1983) Nature 301:634); Adenovirus 5 E2/E1A (Imperiale and Nevins (1984) Mol. Cell. Biol. 4:875); c-jun/Phorbol Ester (TPA), H₂O₂; Collagenase/Phorbol Ester (TPA) (Angel et al. (1987) Mol. Cell. Biol. 7:2256); Stromelysin/Phorbol Ester (TPA), IL-1 (Angel et al. (1987) Cell 49:729); SV40/Phorbol Ester (TPA) (Angel et al. (1987) Cell 49:729); Murine MX Gene/Interferon, Newcastle Disease Virus; GRP78 Gene/A23187 (Resendez Jr. et al. (1988) Mol. Cell. Biol. 8:4579); α-2-Macroglobulin/IL-6; Vimentin/Serum (Kunz et al. (1989) Nucl. Acids Res. 17:1121); MHC Class I Gene H-2 kB/Interferon (Blanar et al. (1989) EMBO J. 8:1139); HSP70/Ela, SV40 Large T Antigen (Taylor and Kingston (1990) Mol. Cell. Biol. 10:165; Taylor and Kingston (1990) Mol. Cell. Biol. 10:176; Taylor et al. (1989) J. Biol. Chem. 264:15160); Proliferin/Phorbol Ester-TPA (Mordacq and Linzer (1989) Genes and Dev. 3:760); Tumor Necrosis Factor/PMA (Hensel et al. (1989) Lymphokine Res. 8:347); Thyroid Stimulating Hormone a Gene/Thyroid Hormone (Chatterjee et al. (1989) Proc. Nat'l Acad. Sci. USA. 86:9114); and, Insulin E Box/Glucose.

Such expression cassettes can be contained in a vector which allow for the introduction of the expression cassette into a cell. In specific embodiments, the vector allows for autonomous replication of the expression cassette in a cell or may be integrated into the genome of a cell. Such vectors are replicated along with the host genome (e.g., nonepisomal mammalian vectors). In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses). See, for example, U.S. Publication 2005214851, herein incorporated by reference.

Any expression cassette can further comprise a selection marker. As used herein, the term “selection marker” comprises any polynucleotide, which when expressed in a cell allows for the selection of the transformed cell with the vector. For example, a selection marker can confer resistance to a drug, a nutritional requirement, or a cytotoxic drug. A selection marker can also induce a selectable phenotype such as fluorescence or a color deposit. A “positive selection marker” allows a cell expressing the marker to survive against a selective agent and thus confers a positive selection characteristic onto the cell expressing that marker. Positive selection marker/agents include, for example, Neo/G418, Neo/Kanamycin, Hyg/Hygromycin, hisD/Histidinol, Gpt/Xanthine, Ble/Bleomycin, HPRT/Hypoxanthine. Other positive selection markers include DNA sequences encoding membrane bound polypeptides. Such polypeptides are well known to those skilled in the art and can comprise, for example, a secretory sequence, an extracellular domain, a transmembrane domain and an intracellular domain. When expressed as a positive selection marker, such polypeptides associate with the cell membrane. Fluorescently labeled antibodies specific for the extracellular domain may then be used in a fluorescence activated cell sorter (FACS) to select for cells expressing the membrane bound polypeptide. FACS selection may occur before or after negative selection.

A “negative selection marker” allows the cell expressing the marker to not survive against a selective agent and thus confers a negative selection characteristic onto the cell expressing the marker. Negative selection marker/agents include, for example, HSV-tk/Acyclovir or Gancyclovir or FIAU, Hprt/6-thioguanine, Gpt/6-thioxanthine, cytosine deaminase/5-fluoro-cytosine, diphtheria toxin or the ricin toxin. See, for example, U.S. Pat. No. 5,464,764, herein incorporated by reference.

In preparing an expression cassette or a homologous recombination cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The host cells of the invention can also be used to produce nonhuman transgenic animals. For example, in one embodiment, a host cell of the invention is a fertilized oocyte or an embryonic stem cell into which a sequence encoding a C-terminal domain deleted ERG polypeptide or a silencing element of the invention has been introduced. Such host cells can then be used to create nonhuman transgenic animals in which exogenous sequences encoding a C-terminal domain deleted ERG polypeptide, a polynucleotide comprising an ERG intragenic deletion, or a polynucleotide expressing a silencing element of the invention have been introduced into their genome or homologous recombinant animals. In specific embodiments, the endogenous ERG sequences have been altered to produce a C-terminal domain deleted ERG polypeptide or to disrupt expression of the entire ERG polypeptide expressed from the ERG intragenic deletion. Such animals are useful for studying the function and/or activity of ERG alleles having intragenic deletions associated with the novel subtype of B-progenitor ALL or encoding C-terminal domain deleted ERG proteins and for identifying and/or evaluating modulators of the activity of the C-terminal domain deleted ERG polypeptide.

As used herein, a “transgenic animal” is a nonhuman animal, in specific embodiments a mammal, a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal As used herein, a “homologous recombinant animal” is a nonhuman animal, in specific embodiments a mammal, in other embodiments a mouse, in which an endogenous ERG gene has been altered by homologous recombination between the endogenous gene and an exogenous DNA molecule introduced into a cell of the animal, e.g., an embryonic cell of the animal, prior to development of the animal

A transgenic animal of the invention can be created by introducing a C-terminal domain deleted ERG polypeptide encoding nucleic acid, a polynucleotide comprising an ERG intragenic deletion, or a polynucleotide expressing a silencing element of the invention into the male pronuclei of a fertilized oocyte, e.g., by microinjection, retroviral infection, and allowing the oocyte to develop in a pseudopregnant female foster animal. Such sequences can be introduced as a transgene into the genome of a nonhuman animal Alternatively, a homologue of the ERG gene can be isolated based on hybridization and used as a transgene. Intronic sequences and polyadenylation signals can also be included in the transgene to increase the efficiency of expression of the transgene. A tissue-specific regulatory sequence(s) can be operably linked to the transgene to direct expression of the sequence particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan (1986) Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the C-terminal domain deleted ERG protein, the polynucleotide comprising and ERG intragenic deletion, or the polynucleotide expressing a silencing element of the invention in its genome and/or expression of mRNA of such sequences in tissues or cells of the animals. A transgenic founder animal can then be used to breed additional animals carrying the transgene. Moreover, transgenic animals carrying a transgene can further be bred to other transgenic animals carrying other transgenes.

To create a homologous recombinant animal, one prepares a vector containing at least a portion of a sequence encoding a C-terminal domain deleted ERG polypeptide or a homolog of the gene into which a deletion has been introduced to thereby allow for the expression of a C-terminal domain deleted ERG polypeptide. In one embodiment, the homologous recombination vector, the altered portion of the ERG gene is flanked at its 5′ and 3′ ends by additional nucleic acid of the ERG gene to allow for homologous recombination to occur between the exogenous ERG gene carried by the vector and an endogenous ERG gene in an embryonic stem cell. The additional flanking ERG nucleic acid is of sufficient length for successful homologous recombination with the endogenous gene. Typically, several kilobases of flanking DNA (at both the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation), and cells in which the introduced ERG gene has homologously recombined with the endogenous ERG gene are selected (see, e.g., Li et al. (1992) Cell 69:915). The selected cells are then injected into a blastocyst of an animal (e.g., a mouse) to form aggregation chimeras (see, e.g., Bradley (1987) in Teratocarcinomas and Embryonic Stem Cells: A Practical Approach, ed. Robertson (IRL, Oxford pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic nonhuman animals containing selected systems that allow for regulated expression of the transgene can be produced. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT Publication Nos. WO 97/07668 and WO 97/07669.

### VII. Antibodies

The present invention further provides antibodies specific to epitopes of the C-terminal domain deleted ERG polypeptide and methods of detecting the C-terminal domain deleted ERG polypeptide, or any combination thereof that rely on the ability of these antibodies to selectively bind to specific portions of the C-terminal domain deleted ERG polypeptide that are unique to that truncated polypeptide. Such antibodies do not bind preferentially to the native or full length ERG polypeptide.

Thus, the C-terminal domain deleted ERG polypeptides of the present invention, including fragments thereof, may be used as immunogens to produce antibodies having use in the diagnostic, research, and therapeutic methods described below. The antibodies may be polyclonal or monoclonal, chimeric, humanized, single chain or Fab fragments. Various procedures known to those of ordinary skill in the art may be used for the production and labeling of such antibodies and fragments. See, e.g., Burns, ed., Immunochemical Protocols, 3.sup.rd ed., Humana Press (2005); Harlow and Lane, Antibodies: A Laboratory Manual, Cold Spring Harbor Laboratory (1988); Kozbor et al., Immunology Today 4: 72 (1983); Kohler and Milstein, Nature 256: 495 (1975). Antibodies or fragments exploiting the differences between the C-terminal domain deleted ERG polypeptide and the native or full length ERG polypeptide are particularly preferred.

As discussed elsewhere herein, methods are provided for detecting the presence of the C-terminal domain deleted ERG polypeptide. Such antibodies can be used to detect the presence of the fusion protein in samples from human cells. The methods of the invention involve the use of antibodies that bind to a C-terminal domain deleted ERG polypeptide and antibody detection systems that are known to those of ordinary skill in the art. Such methods find use in diagnosis and treatment of ALL, for example, to determine if particular cells or tissues express the C-terminal domain deleted ERG polypeptide.

Conditions for incubating an antibody with a test sample vary depending on the format employed for the assay, the detection methods employed, the nature of the test sample, and the type and nature of the antibody used in the assay. One skilled in the art will recognize that any one of the commonly available immunological assay formats (such as radioimmunoassays, enzyme-linked immunosorbent assays, diffusion based ouchterlony, or rocket inmunofluorescent assays) can readily be adapted to employ the antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of Enzyme Immunoassays: Laboratory Techniques in Biochemistry and Molecular Biology, Elsevier Science Publishers, Amsterdam, The Netherlands (1985).

In another embodiment of the immunoassays of the invention, the anti-N-terminal domain deleted ERG polypeptide antibody is immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling antibodies to such solid supports are well known in the art (see, for example, Weir, D. M. et al., Handbook of Experimental Immunology, 4th Ed., Blackwell Scientific Publications, Oxford, England, Chapter 10 (1986)).

Additionally, one or more of the antibodies used in the above described methods can be detectably labeled prior to use. Antibodies can be detectably labeled through the use of radioisotopes, affinity labels (such as biotin, avidin, etc.), enzymatic labels (such as horse radish peroxidase, alkaline phosphatase, etc.) fluorescent labels (such as FITC or rhodamine, etc.), paramagnetic atoms, etc. Procedures for accomplishing such labeling are well-known in the art; see, for example, Sternberger, L. A. et al., J. Histochem. Cytochem. 18:315-333 (1970); Bayer, E. A. et al., Meth. Enzym. 62:308-315 (1979); Engrall, E. et al., J. Immunol. 109:129-135 (1972); Goding, J. W., J. Immunol. Meth. 13:215-226 (1976).

### VIII. Pharmaceutical Compositions

The methods and compositions of the invention find use in the treatment or prevention of leukemia, more specifically, to the treatment or prevention of the novel subtype of B-progenitor ALL. Such methods comprise the administration of an agent that blocks the activity or the level of expression of a C-terminal domain truncated ERG polypeptide.

The therapeutic agent may further comprise an inorganic or organic, solid or liquid, pharmaceutically acceptable carrier. The carrier may also contain preservatives, wetting agents, emulsifiers, solubilizing agents, stabilizing agents, buffers, solvents and salts. Compositions may be sterilized and exist as solids, particulates or powders, solutions, suspensions or emulsions.

The therapeutic agent can be formulated according to known methods to prepare pharmaceutically useful compositions, such as by admixture with a pharmaceutically acceptable carrier vehicle. Suitable vehicles and their formulation are described, for example, in Remington's Pharmaceutical Sciences (16th ed., Osol, A. (ed.), Mack, Easton Pa. (1980)). In order to form a pharmaceutically acceptable composition suitable for effective administration, such compositions will contain an effective amount of the therapeutic agent, either alone, or with a suitable amount of carrier vehicle.

The pharmaceutically acceptable carrier will vary depending on the method of administration and the intended method of use. The pharmaceutical carrier employed may be, for example, either a solid, liquid, or time release. Representative solid carriers are lactose, terra alba, sucrose, talc, gelatin, agar, pectin, acacia, magnesium stearate, stearic acid, microcrystalin cellulose, polymer hydrogels, and the like. Typical liquid carriers include syrup, peanut oil, olive oil, cyclodextrin, and the like emulsions. Those skilled in the art are familiar with appropriate carriers for each of the commonly utilized methods of administration. Furthermore, it is recognized that the total amount of the therapeutic agent administered will depend on both the pharmaceutical composition being administered (i.e., the carrier being used), the mode of administration, binding activity and the desired effect (i.e., a method of detecting, a method of modulating, or a method of delivering a therapeutic agent).

Once the pharmaceutical composition has been formulated, it may be stored in sterile vials as a solution, suspension, gel, emulsion, solid, or dehydrated or lyophilized powder. Such formulations may be stored either in a ready to use form or requiring reconstitution immediately prior to administration.

The therapeutic agent also can be delivered locally to the appropriate cells, tissues or organ system by using a catheter or syringe. Other means of delivering such therapeutic agents locally to cells include using infusion pumps (for example, from Alza Corporation, Palo Alto, Calif.) or incorporating the therapeutic agent into polymeric implants (see, for example, Johnson eds. (1987) Drug Delivery Systems (Chichester, England: Ellis Horwood Ltd.), which can affect a sustained release of the therapeutic agent to the immediate area of the implant.

A variety of methods are available for delivering a therapeutic agent to a subject (i.e., an animal (mammal), tissue, organ, or cell). The manner of administering therapeutic agents for systemic delivery may be via subcutaneous, ID, intramuscular, intravenous, or intranasal. In addition inhalant mists, orally active formulations, transdermal iontophoresis or suppositories, are also envisioned. One carrier is physiological saline solution, but it is contemplated that other pharmaceutically acceptable carriers may also be used. In one embodiment, it is envisioned that the carrier and the therapeutic agent constitute a physiologically-compatible, slow release formulation. The primary solvent in such a carrier may be either aqueous or non-aqueous in nature. In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption of the therapeutic agent. Such excipients are those substances usually and customarily employed to formulate dosages for parental administration in either unit dose or multi-dose form.

For example, in general, the disclosed therapeutic agent can be incorporated within or on microparticles or liposomes. Microparticles or liposomes containing the disclosed therapeutic agent can be administered systemically, for example, by intravenous or intraperitoneal administration, in an amount effective for delivery of the therapeutic agent to targeted cells. Other possible routes include trans-dermal or oral administration, when used in conjunction with appropriate microparticles. Generally, the total amount of the liposome-associated therapeutic agent administered to an individual will be less than the amount of the unassociated therapeutic agent that must be administered for the same desired or intended effect.

By “ effective amount” is meant the concentration of a therapeutic agent that is sufficient to elicit a desired effect (i.e., the treatment or prevention of leukemia).

Thus, the concentration of a therapeutic agent in an administered dose unit in accordance with the present invention is effective to produce the desired effect. The effective amount will depend on many factors including, for example, the responsiveness of the subject, the weight of the subject along with other intrasubject variability, the method of administration, and the formulation used. Methods to determine efficacy, dosage, Ka, and route of administration are known to those skilled in the art.

Thus the present invention also provides pharmaceutical formulations or compositions, both for veterinary and for human medical use, which comprise the therapeutic agent with one or more pharmaceutically acceptable carriers thereof and optionally any other therapeutic ingredients. The carrier(s) must be pharmaceutically acceptable in the sense of being compatible with the other ingredients of the formulation and not unduly deleterious to the recipient thereof.

The compositions include those suitable for oral, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier or both, and then, if necessary, shaping the product into desired formulations.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parental administration conveniently comprise a sterile aqueous preparation of the active compound, which can be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Further, the present invention provides liposomal formulations of the therapeutic agent. The technology for forming liposomal suspensions is well known in the art. When the therapeutic agent is an aqueous-soluble salt, using conventional liposome technology, the same may be incorporated into lipid vesicles. In such an instance, due to the water solubility of the compound, the compound will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer that forms the structure of the liposome. In either instance, the liposomes that are produced may be reduced in size, as through the use of standard sonication and homogenization techniques. The liposomal formulations containing the progesterone metabolite or salts thereof, may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the desired therapeutic agent or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts.

In addition to the aforementioned ingredients, the compositions of the invention may further include one or more accessory ingredient(s) selected from the group consisting of diluents, buffers, flavoring agents, binders, disintegrants, surface active agents, thickeners, lubricants, preservatives (including antioxidants) and the like.

The following examples are offered by way of illustration and not by way of limitation.

### Experimental

ERG Deletions define a Novel Subtype of B-Progenitor Acute Lymphoblastic Leukemia

In a previous gene expression profiling study of acute lymphoblastic leukemia (ALL), a novel subtype of B-progenitor ALL (4.9% of 284 cases) with a unique gene expression profile, aberrant expression of CD2 and the absence of recurring cytogenetic abnormalities was identified (Yeoh et al. (2002) Cancer Cell 1:133). Efforts to identify rearrangement or mutation of many of the top-ranked genes in the novel expression signature, including PDGFRA, PTPRM, BRDG1, LHFPL2, and CHST2 failed to identify a causative lesion. To further investigate the genetic basis of this subtype, we have performed integrated genomic analysis of 277 ALL cases. Affymetrix Mapping 250 k Sty and Nsp single nucleotide polymorphism microarrays were used in all cases, and Affymetric U133A gene expression profiles were obtained on 183 of the cases. Unsupervised clustering of gene expression data identified 16 cases of the novel subtype in this expanded patient cohort, included all of the 14 cases previously reported in the study of Yeoh et al. (2002) Cancer Cell 1:133. Remarkably, focal mono-allelic deletions of the ETS family member ERG (v-ets erythroblastosis virus E26 oncogene homolog) were detected by genome-wide copy number analysis in 12/16 (75%) of the novel cases, but not in any other ALL subtype. An extensive analysis failed to reveal any evidence of translocations involving the altered ERG allele, indicating that these are intragenic deletions limited exclusively to ERG. The presence and extent of the deletions was confirmed by genomic quantitative PCR. The ERG deletions involved a subset of internal exons (most commonly genomic ERG exons 6-10 or 6-12) and resulted in the expression of internally deleted ERG transcripts with altered reading frames that are predicted to produce a prematurely truncated N-terminal protein fragment; however, using an alternative translational start site 5′ to exon 13, the transcripts should also encode a ˜28 kDa C-terminal ERG fragment that contains the entire C-terminal ETS DNA-binding and transactivation domains, but lacks all N-terminal domains. Importantly, western blot analysis of primary leukemic blasts revealed expression of only the 28 kDa C-terminal ERG protein, along with full length ERG expressed from the retained wild type allele. Remarkably, the C-terminal ERG protein was also detected in 3 of 4 novel ALL cases that lacked detectable ERG deletions, but not in any other ALL subtype. In luciferase reporter assays, the aberrant ERG protein acted as a competitive (dominant negative) inhibitor of wild type ERG. Analysis of a second cohort of 35 additional B-progenitor ALL cases lacking recurring cytogenetic abnormalities identified two cases with ERG deletions and a third expressing the aberrant ERG protein, all of which had the novel gene expression profile. Notably, resequencing of ERG in 252 ALL cases identified only one case with an ERG mutation that resulted in a frameshift in the ETS domain. This case did not share the novel signature nor express the aberrant C-terminal ERG protein. Importantly, this case harbored three copies of chromosome 21, and thus had two normal copies of the ERG gene. Furthermore, in contrast to the deletional forms of ERG observed in novel ALL, the sequence mutation in this case is predicted to result in loss of the C-terminal ERG domains, and so is predicted to be hypomorphic, and not act as a competitive (dominant negative) inhibitor of normal ERG. Finally, in an analysis of 37 acute leukemia cell lines, the B-progenitor ALL line NALM-6 was found to harbor a focal, internally truncating ERG deletion, expressed the aberrant ERG protein, and shared the novel gene expression profile, thus identifying it as a model of this novel ALL subtype. These data establish focal ERG deletions as the genetic lesion underlying a novel subtype of ALL, and have expanded the genetic mechanisms that lead to the dysregulation of ERG transcriptional activity from chromosomal translocations that result in enhanced transcriptional activity (e.g. TMPRSS2-ERG observed in carcinoma of the prostate), to deletions that generate dominant negative forms of the transcriptional factor.

TABLE 1
Summary of SEQ ID NOS.

| SEQ ID NO | Type | Description |
|---|---|---|
| 1 | Genomic | Genomic DNA comprising the wild-type ERG gene. |
| | DNA | |
| 2 | cDNA | cDNA of ERG1 isoform |
| 3 | cDNA | cDNA of ERG2 isoform |
| 4 | cDNA | ERGΔexon 6-10 |
| 5 | cDNA | ERGΔexon 6-12 |
| 6 | cDNA | ERGΔexon 6-13 |
| 7 | cDNA | junction of the intragenic deletion of exons 6-10 of |
| | | the ERGΔexon 6-10 cDNA (20 nucleotides 5′ and 20 |
| | | nucleotides 3′ of the junction.) |
| 8 | cDNA | junction of the intragenic deletion of exons 6-10 of |
| | | the ERGΔexon 6-10 cDNA (50 nucleotides 5′ and 50 |
| | | nucleotides 3′ of the junction.) |
| 9 | cDNA | junction of the intragenic deletion of exons 6-10 of |
| | | ERGΔexon 6-10 cDNA (100 nucleotides 5′ and 100 |
| | | nucleotides 3′ of the junction.) |
| 10 | cDNA | junction of the intragenic deletion of exons 6-10 of |
| | | ERGΔexon 6-10 cDNA (150 nucleotides 5′ and 150 |
| | | nucleotides 3′ of the junction.) |
| 11 | cDNA | junction of the intragenic deletion of exons 6-12 of |
| | | ERGΔexon 6-12 cDNA (20 nucleotides 5′ and 20 |
| | | nucleotides 3′ of the junction.) |
| 12 | cDNA | junction of the intragenic deletion of exons 6-12 of |
| | | ERGΔexon 6-12 cDNA (50 nucleotides 5′ and 50 |
| | | nucleotides 3′ of the junction.) |
| 13 | cDNA | junction of the intragenic deletion of exons 6-12 of |
| | | ERGΔexon 6-12 cDNA (100 nucleotides 5′ and 100 |
| | | nucleotides 3′ of the junction.) |
| 14 | cDNA | junction of the intragenic deletion of exons 6-12 of |
| | | ERGΔexon 6-12 cDNA (150 nucleotides 5′ and 150 |
| | | nucleotides 3′ of the junction.) |
| 15 | Polypeptide | wild-type ERG protein encoded by ERG1 isoform |
| 16 | Polypeptide | ERG_I1_D6-10_distal ORF |
| 17 | Polypeptide | ERG_I1_D6-12_distal ORF |
| 18 | Polypeptide | TMP_ERGa_ERG1_CDS |
| 19 | Genomic | Genomic DNA ERGD6-10 |
| 20 | polypeptide | ERG_I2_D6-12_distal ORF |
| 21 | cDNA | junction of the intragenic deletion of exons 6-10 of |
| | | the ERGΔexon 6-10 cDNA (10 nucleotides 5′ and 10 |
| | | nucleotides 3′ of the junction.) |
| 22 | cDNA | junction of the intragenic deletion of exons 6-12 of |
| | | ERGΔexon 6-12 cDNA (10 nucleotides 5′ and 10 |
| | | nucleotides 3′ of the junction.) |
| 27 | cDNA | junction of the intragenic deletion of exons 6-13 of |
| | | ERGΔexon 6-13 cDNA (10 nucleotides 5′ and 10 |
| | | nucleotides 3′ of the junction.) |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated polynucleotide comprising a nucleotide sequence having at least 95% sequence identity to SEQ ID NO: 21, 22 or 6, wherein the presence of said polynucleotide in the nucleic acid complement of a biological sample is indicative of a novel subtype of B-progenitor ALL.

2. The isolated polynucleotide of claim 1, wherein said polynucleotide comprises the sequence set forth in SEQ ID NO: 21 or 22.

3. The isolated polynucleotide of claim 1, wherein said polynucleotide comprises a nucleotide sequence having at least 95% sequence identity to the sequence set forth in SEQ ID NO:7, 8, 9, 10, 11, 12, 13, 14.

4. The isolated polynucleotide of claim 3, wherein said polynucleotide comprises the sequence set forth in SEQ ID NO: 4 or 5.

5. An isolated polypeptide comprising an amino acid sequence having at least 90% sequence identity to the amino acid sequence set forth in SEQ ID NO: 16 or 17, wherein said polypeptide does not contain a DNA binding PNT domain and a CAE domain and said polypeptide has dominant negative ERG activity.

6. The isolated polypeptide of claim 5, wherein said polypeptide comprises the amino acid sequence set forth in SEQ ID NO: 16 or 17.

7. A kit for detecting a novel subtype of B-progenitor acute lymphoblastic leukemia (ALL) in a biological sample comprising a reagent comprising a polynucleotide that can detect an intragenic deletion in the ERG gene in the nucleic acid complement of said biological sample, wherein said intragenic deletion comprises the deletion of at least exons 6-10 of the ERG gene.

8. The kit of claim 7, wherein the intragenic deletion comprises the deletion of exons 6-12 of the ERG gene.

9. The kit of claim 7, wherein the intragenic deletion comprises the deletion of exons 6-13 of the ERG gene.

10. The kit of claim 7, wherein said reagent detects said intragenic deletion by directly assaying the genomic DNA sequence.

11. The kit of claim 7, wherein said reagent detects said intragenic deletion or by directly assaying the transcript produced from the genomic DNA.

12. The kit of claim 7, wherein said reagent comprises a pair of primers that amplify an amplicon comprising the sequence set forth in SEQ ID NO: 21, 22 or 6 or a polynucleotide having at least 95% sequence identity to SEQ ID NO: 21, 22 or 6 and thereby detect the intragenic deletion of the ERG gene.

13. The kit of claim 7, wherein said reagent comprises at least one probe comprising a polynucleotide sequence that hybridizes under stringent conditions to said ERG gene and thereby detects the intragenic deletion of the ERG gene.

14. The kit of claim 13, wherein said probe comprises the sequence set forth in SEQ ID NO: 21 or 22 or a polynucleotide having at least 95% sequence identity to SEQ ID NO: 21 or 22.

15. A method for assaying a biological sample for an intragenic deletion of a v-ets erythroblastosis virus E26 oncogene homolog (ERG) gene comprising detecting the intragenic deletion in the nucleic acid complement of said biological sample, wherein the presence of said intragenic deletion is indicative of a novel subtype of B-progenitor ALL.

16. A method for diagnosing a novel subtype of B-progenitor ALL in a leukemia patient comprising assaying a biological sample for an intragenic deletion of a v-ets erythroblastosis virus E26 oncogene homolog (ERG) gene comprising detecting the intragenic deletion in the nucleic acid complement of said biological sample, wherein the presence of said intragenic deletion is indicative of the novel subtype of B-progenitor ALL.

17. The method of claim 16, further comprising selecting a therapy for said patient.

18. The method of claim 15, wherein the intragenic deletion results in the expression of a C-terminal domain deleted ERG polypeptide having dominant negative ERG activity.

19. The method of claim 18, wherein said intragenic deletion of the ERG gene comprises the deletion of exon 6 through exon 10 of the ERG gene.

20. The method of claim 18, wherein said intragenic deletion of the ERG gene comprises the deletion of exon 6 through exon 12 of the ERG gene.

21. The method of claim 16, wherein said intragenic deletion of the ERG gene comprises the deletion of exon 6 through exon 13 of the ERG gene.

22. The method of claim 15, wherein determining if said biological sample comprises the intragenic deletion comprises a nucleic acid sequencing technique.

23. The method of claim 15, wherein determining if said biological sample comprises the intragenic deletion comprises a nucleic acid hybridization technique.

24. The method of claim 23, wherein said nucleic acid hybridization technique is selected from the group consisting of in situ hybridization (ISH), microarray, and Southern blot.

25. The method of claim 23, wherein said nucleic acid hybridization technique comprises a probe comprising the sequence set forth in SEQ ID NO: 21 or 22 or a polynucleotide having at least 95% sequence identity to SEQ ID NO: 21 or 22.

26. The method of claim 15, wherein said reagent detects said intragenic deletion by directly assaying the genomic DNA sequence.

27. The method of claim 15, wherein said reagent detects said intragenic deletion or by directly assaying the transcript produced from the genomic DNA.

28. The method of claim 15, wherein determining if said biological sample comprises the intragenic deletion comprises a nucleic acid amplification method.

29. The method of claim 28, wherein said nucleic acid amplification method comprises polymerase chain reaction (PCR), reverse transcription polymerase chain reaction (RT-PCR), transcription-mediated amplification (TMA), ligase chain reaction (LCR), strand displacement amplification (SDA) and nucleic acid sequence based amplification (NASBA).

30. The method of claim 29, wherein said nucleic acid amplification method amplifies a polynucleotide set forth in SEQ ID NO: 21, 22 or 6 or a polynucleotide having at least 95% sequence identity to SEQ ID NO: 21, 22 or 6.

31. The method of claim 15, wherein said biological sample is selected from the group consisting of peripheral blood, bone marrow, apheresis samples, cerebrospinal fluid, saliva, urine, gonadal tissue, tissue (e.g. chloroma) biopsies, or any other human tissue sample potentially involved by leukemic infiltration.

32. The method of claim 15, wherein said biological sample is from a human.

33. A method for assaying a biological sample for an C-terminal domain deleted v-ets erythroblastosis virus E26 oncogene homolog (ERG) polypeptide comprising providing a biological sample and detecting said C-terminal domain deleted ERG polypeptide in said sample, wherein the presence of said C-terminal domain deleted ERG polypeptide is indicative of a novel subtype of B-progenitor ALL.

34. A method for diagnosing a novel subtype of B-progenitor ALL in a leukemia patient comprising providing a biological sample and assaying said biological sample for an C-terminal domain deleted v-ets erythroblastosis virus E26 oncogene homolog (ERG) polypeptide, wherein the presence of said C-terminal domain deleted ERG polypeptide is indicative of the novel subtype of B-progenitor ALL.

35. The method of claim 33, wherein said C-terminal domain deleted ERG polypeptide comprising the amino acid sequence set forth in SEQ ID NO: 16 or 17.

36. The method of claim 33, wherein said C-terminal domain deleted ERG polypeptide comprises an amino acid sequence having at least 90% sequence identity to SEQ ID NO: 16 or 17, wherein said C-terminal domain deleted ERG polypeptide does not contain a DNA binding PNT domain and a CAE domain and said polypeptide has dominant negative ERG activity.

37. The method of claim 34, further comprising selecting a therapy for said patient.

38. The method of claim 33, wherein said biological sample is selected from the group consisting of peripheral blood, bone marrow, apheresis samples, cerebrospinal fluid, saliva, urine, gonadal tissue, tissue (e.g. chloroma) biopsies, or any other human tissue sample potentially involved by leukemic infiltration.

39. The method of claim 33, wherein said biological sample is from a human.

40. A non-human transgenic animal that has been altered to express a polypeptide comprising an amino acid sequence having at least 90% sequence identity to the amino acid sequence set forth in SEQ ID NO: 17, wherein said polypeptide does not contain a DNA binding PNT domain and a CAE domain and said polypeptide has dominant negative ERG activity.

41. The non-human transgenic animal of claim 40, wherein said polypeptide comprises the amino acid sequence set forth in SEQ ID NO: 16 or 17.

42. The non-human transgenic animal of claim 40, wherein said polypeptide is encoded by a polynucleotide comprising an intragenic deletion of the ERG gene.

43. The non-human transgenic animal of claim 42, wherein said polynucleotide comprises a deletion of exons 6-10 of the ERG gene.

44. The non-human transgenic animal of claim 42, wherein said polynucleotide comprises a deletion of exons 6-12 of the ERG gene.

45. A method of screening for agents capable of selectively inhibiting the activity of a C-terminal-domain deleted ERG polypeptide having dominant negative ERG activity comprising:
a) contacting said compound with the C-terminal-domain deleted ERG polypeptide, and
b) determining whether said compound inhibits the activity of said C-terminal-truncated ERG polypeptide.

46. The method of claim 45, wherein
said C-terminal-truncated ERG polypeptide is expressed in a eukaryotic cell; and,
determining whether said compound inhibits the activity of said C-terminal-domain deleted ERG polypeptide comprises monitoring said cell for a suppression or elimination of an adverse phenotype associated with expression of the C-terminal-domain deleted ERG polypeptide; wherein an agent which suppresses or eliminates said adverse phenotype is identified as an inhibitor of the C-terminal-domain deleted ERG polypeptide.

47. A method of identifying an agent capable of selectively binding a C-terminal domain deleted ERG polypeptide having the dominant negative ERG activity comprising the steps of:
(a) contacting a candidate agent with said C-terminal domain deleted ERG polypeptide; and,
(b) determining whether said candidate agent specifically binds said C-terminal domain deleted ERG polypeptide.

48. The method of claim 45 wherein said polypeptide comprising an amino acid sequence having at least 90% sequence identity to the amino acid sequence set forth in SEQ ID NO: 16 or 17, wherein said polypeptide does not contain a DNA binding PNT domain and a CAE domain and said polypeptide has dominant negative ERG activity.
